(19) United States
(12) Patent Application Publication
(10) Pub. No.: US 2002/0098516 A1
(43) Pub. Date: Jul. 25, 2002

(54) Immunodiagnostic determination of usher syndrome type IIA

(75) Inventor: Dominic E. Cosgrove, Omaha, NE (US)

Correspondence Address: MUETING, RAASCH & GEBHARDT, P.A., P.O. BOX 581415, MINNEAPOLIS, MN, 55458, US

(73) Assignee: BOYSTOWN NATIONAL RESEARCH HOSPITAL

(21) Appl. No.: 09/970,318

(22) Filed: Oct. 3, 2001

Related U.S. Application Data
(60) Provisional application No. 60/237,834, filed on Oct. 3, 2000.

(51) Int. Cl. G01N 33/53

(52) U.S. Cl. 435/7.1

(57) ABSTRACT

Methods and test kits for determining whether an individual has or is at risk for developing Usher syndrome Type IIa. The methods include obtaining a biological sample from an individual, incubating the biological sample with at least one antibody which is immunoreactive with an USH2a protein under conditions effective to produce immunoconjugates if the usherin protein is present, evaluating for the presence or absence of immunoconjugates, and correlating the presence of immunoconjugates with the individual not having Usher syndrome Type IIa, and the absence of immunoconjugates with the individual having or being at risk for developing Usher syndrome Type IIa.

This application claims the benefit of U.S. Provisional Application Ser. No. 60/237,834, filed Oct. 3, 2000, which is incorporated herein by reference in its entirety.

### STATEMENT OF GOVERNMENT RIGHTS

This invention was made with government support under grants from the United States Department of Health and Human Services, National Institutes of Health, National Institute on Deafness and other Disorders of Communication, Grant No. RTC P60 DC00982. The U.S. government may have certain rights in this invention.

### BACKGROUND OF THE INVENTION

Usher syndrome is the leading genetic disorder of combined blindness and deafness after childhood. The main clinical symptoms of the disease are retinitis pigmentosa (RP) and hearing loss. Affected individuals have a sensorineural hearing impairment at birth and later develop progressive visual impairment secondary to RP. Vestibular dysfunction is also, in some cases, a feature of the syndrome.

The frequency of Usher syndrome has been estimated at 3.0/100,000 in Scandinavia and at 4.4/100,000 in the United States. Overall, there are about 20,000 deaf and blind people in the United States, of whom more than half are believed to have Usher syndrome. Conversely, the frequency of deafness in the RP population is estimated to range from 18.0 to 33.3%.

Usher syndrome Type II is the most common of the three Usher syndromes. Although originally it was believed that Usher Type II accounted for only about 10% of all Usher cases, more recent research shows that Type II actually accounts for over half of all Usher cases. The USH2A gene has been localized to chromosome 1q41 between D1S474 and AFM144FX2 (Kimberling et al., Am. J. Hum. Genet., 56:216-223 (1995); Sumegi et al., Genomics, 35:79-86 (1996)), and more recently, the gene has been identified (Eudy et al., Science, 280:1753-1757 (1998)). However, there are Usher Type II families whose disease locus cannot be linked to the 1q41 region. Two new Usher II loci have been localized to 3p and 5q (Pieke-Dashl et al., J. Med. Genet., 37:256-262 (2000); Hmani et al., Eur. J. Hum. Genet., 7:363-367 (1999)). These new genes have been given the designation USH2B and USH2C, leaving USH2A to refer to the original 1q41 locus.

Currently there is no definitive diagnostic technique available to determine whether a person has Usher syndrome Type IIa. Diagnosis is based on clinical evaluations, and thus requires the development of the phenotype, precluding early treatment. These subjective examinations are also fraught with inherent uncertainty. Thus, there is a need for an assay for determining the presence or absence of the protein in tissues as a diagnostic procedure aimed at early diagnosis of Usher syndrome Type IIa.

### SUMMARY OF THE INVENTION

The present invention provides a method of determining whether an individual has or is at risk for developing Usher syndrome Type IIa. The method includes: obtaining a biological sample from the individual; incubating the biological sample with at least one antibody which is immunoreactive with at least a portion of a human usherin protein under conditions effective to produce an immunoconjugate if the usherin protein is present, wherein a complement of a polynucleotide encoding the usherin protein is capable of hybridizing to the polynucleotide represented by SEQ ID NO:3 under highly stringent hybridization conditions; evaluating for the presence or absence of the immunoconjugate; and correlating the presence of the immunoconjugate with the individual not having Usher syndrome Type IIa, and the absence of the immunoconjugate with the individual having or being at risk for developing Usher syndrome Type IIa.

In a preferred embodiment of the present invention, the biological sample is selected from the group consisting of at least a portion of testis, cochlea, epididymus, ovary, eye, uterus, heart, pancreas, prostate, skin, placenta, spleen, submaxillary gland, small intestine, large intestine, blood vessels, and combinations thereof. In another preferred embodiment of the present invention, the at least one antibody is a monoclonal, polyclonal, or combinations thereof, that has an attached detectable label, which can include radioactive, nonradioactive, and other detectable molecules known in the art. Combinations of such labels can be used if desired. Optionally, the at least one antibody may immunoreact with a polypeptides selected from the group consisting of SEQ ID NO:1, SEQ ID NO:2, SEQ ID NO:4, and combinations thereof. The usherin protein may be encoded by a polynucleotide represented by SEQ ID NO:3.

Another embodiment of the present invention provides a method for detecting the presence or absence of usherin protein. The method includes: obtaining a biological sample; incubating the biological sample with at least one antibody which is immunoreactive with at least a portion of a human usherin protein under conditions effective to produce an immunoconjugate if the usherin protein is present, wherein a complement of a polynucleotide encoding the usherin protein is capable of hybridizing to the polynucleotide represented by SEQ ID NO:3 under highly stringent hybridization conditions; evaluating for the presence or absence of the immunoconjugate; and correlating the presence of the immunoconjugate with the presence of the usherin protein, and the absence of the immunoconjugate with the absence of the usherin protein.

Another embodiment of the present invention provides a method of determining whether an individual has or is at risk for developing Usher syndrome Type IIa. The method includes: obtaining a biological sample from the individual; incubating the biological sample with a first antibody and a second antibody that are immunoreactive with at least a portion of a human usherin protein under conditions effective to produce an immunoconjugate if the usherin protein is present, wherein a complement of a polynucleotide encoding the usherin protein is capable of hybridizing to the polynucleotide represented by SEQ ID NO:3 under highly stringent hybridization conditions; evaluating for the presence or absence of the irnmunoconjugate; and correlating the presence of the immunoconjugate with the individual not having Usher syndrome Type IIa, and the absence of the immunoconjugate with the individual having or being at risk for developing Usher syndrome Type IIa.

In a preferred embodiment of this invention, the biological sample is incubated with an antibody that is immunoreactive with the usherin protein and attached to a solid surface. The usherin protein, if present in the sample, is allowed to immunoreact with the attached antibody and with a second antibody that is immunoreactive with another region of the usherin protein (i.e., a region other than the region immunoreactive with the solid support-attached antibody). The resultant two antibodies-usherin protein complex thereby forms a sandwich. The amount of bound second antibody is detected. This amount of detected second antibody is directly proportional to the amount of attached usherin protein. The presence of usherin protein is indicative of an individual not having Usher syndrome Type IIa. On the other hand, the absence of usherin protein is indicative of an individual having or being at risk for developing Usher syndrome Type IIa.

Another embodiment of the present invention is a test kit that contains an antibody and a detectably-labeled usherin protein to be used in the assay for Usher syndrome Type IIa-diagnostic protein for detecting the presence or absence of Usher syndrome Type IIa in an individual. The monoclonal or polyclonal antibody is attached to a solid support, such as a monoclonal antibody that is coated onto a 96-well microtiter plate. The biological sample is contacted with the antibody attached to the solid support under conditions that allow the at least a portion of usherin protein, if it is present in the sample, to bind to the antibody attached to the solid support, wherein a complement of a polynucleotide encoding the usherin protein is capable of hybridizing to the polynucleotide represented by SEQ ID NO:3 under highly stringent hybridization conditions. A known amount of labeled usherin protein, for example, with biotin or horseradish peroxidase (HRP) or other labels known in the art, is added simultaneously with or subsequent to the addition of the biological sample. The labeled usherin protein attempts to bind to the antibody, however, the labeled usherin protein is inhibited from binding to the antibody by the presence of previously bound nonlabeled usherin protein from the sample. In this way the amount of unlabeled usherin protein in the sample can be measured. The amount of unlabeled usherin protein in the sample is inversely proportional to the signal generated by the labelled usherin protein.

Another embodiment of the present invention is a test kit that contains an antibody to be used in the assay for Usher syndrome Type IIa-diagnostic protein for detecting the presence or absence of Usher syndrome Type IIa in an individual. One of the antibodies is immunoreactive with one epitopic region of at least a portion of an usherin protein and, if a second antibody is included, the second antibody is immunoreactive with an epitopic region of at least a portion of an usherin protein separate from the epitopic region that is immunoreactive with the first antibody, wherein a complement of a polynucleotide encoding the usherin protein is capable of hybridizing to the polynucleotide represented by SEQ ID NO:3 under highly stringent hybridization conditions. In a preferred embodiment of the test kit, there are two antibodies that are immunoreactive with two epitopic regions of the at least a portion of usherin protein. One of the antibodies is attached to a solid support, such as the walls and bottoms of wells of a microtiter plate. The other antibody has a detection label bound to it.

Still another embodiment of the present invention is an antibody that immunoreacts with at least a portion of human usherin protein under conditions effective to produce an immunoconjugate if the usherin protein is present, wherein the absence of an immunoconjugate correlates to the diagnosis of or the individual being at risk for developing Usher Type IIa syndrome, wherein a complement of a polynucleotide encoding the usherin protein is capable of hybridizing to the polynucleotide represented by SEQ ID NO:3 under highly stringent hybridization conditions. The antibody may be a monoclonal antibody, a polyclonal antibody, or combinations thereof.

### BRIEF DESCRIPTION OF THE FIGURES

FIG. 1 illustrates the major structural elements of the usherin protein based on amino acid sequence. The amino acid positions where domains start and end are indicated. The location of polypeptides used to derive antibodies 1 (SEQ ID NO:1) and 2 (SEQ ID NO:2) used in these studies are shown. Constructs used to generate fusion peptides comprised the indicated portions of the LN, LE, and fibronectin type III domains (LN-FP, LE-FP, and FN-FP, respectively).

FIG. 2 is a Western blot of immunoprecipitated protein from extracts of retina and cochlea. For both gels: lane 1 is retinal extract; lane 2 is retinal extract immunoprecipitated with pre-immune serum; lane 3 is cochlear extract; lane 4 is cochlear extract precipitated with pre-immune serum. For the gel on the left, lanes 1 and 3 were immunoprecipitated with antibody 2 and blot probed with antibody 1. For the gel in the right, lane 1 and 3 were immunoprecipitated with antibody 1 and the blot was probed with antibody 2.

FIG. 3 is commercially available PolyA+ RNA dot blot from various mouse tissues. The blot was hybridized to a cDNA fragment corresponding to the LN domain of the protein. The template on the right indicates the tissues from which the corresponding RNA spot on the left was prepared.

FIG. 4 is an immunoperoxidase detection of tissues where usherin is expressed. A survey for usherin expression was conducted on mouse tissues. This figure summarizes where usherin was expressed. Serial sections were stained with hematoxylin and eosin (H&E) to illustrate tissue architecture, or with anti-usherin (left panels), or anti-collagen α1 (IV), which specifically localizes to the basement membranes. Arrows indicate usherin in the capillary basement membranes of the epididymus (D) and the spleen (J). Epidid=epididymus; Submax=submaxillary gland; Sm int=small intestine.

FIG. 5 is an immunoperoxidase detection of tissues where usherin is not expressed. Serial section were stained with hematoxylin and eosin (H&E) to illustrate tissue architecture, or with anti-usherin (left panels), or anti-collagen α1 (IV), which specifically localizes to the basement membranes. Sk musc=skeletal muscle; Sm musc=smooth muscle. Magnification bars are 50 μm.

FIG. 6 is an expression of usherin in the inner ear and the eye of the mouse, and in the human retina. Mid-modiolar cross sections of the adult (8 wks) cochlea (A, B, C), or post-natal day 0 cochlea (G, H, I), or cross sections of adult retina (D, E, F) were immunostained with anti-usherin antibodies (A, D, G) or anti-type IV collagen antibodies (C, F, E). Eosin and hematoxylin stained serial sections are illustrated to provide a cellular frame of reference (B, E, H). Arrows in A and C denote the strial capillary basement membranes, and arrows in D, E, and F denote immunostaining in the basement membranes in Bruch's layer of the retina. Panel J shows expression of the usherin protein in the Bruch's layer and the choroid capillaries in human retina. Human retina was immunostained using the anti-usherin (raised against the mouse protein) antibody. Arrow heads indicate linear immunostaining in the basement membranes on either limiting side of the Bruch's layer (BL). RPE=retinal pigment epithelial side; CL=choroid layer. Magnification bars are 50 μm.

FIG. 7 is an immunogold localization of usherin to the basement membranes in strial capillaries, and the basement membrane in Bruc's layer of the retina. Arrows indicate immunogold particle deposition in the strial capillary basement membranes (A) and the basement membranes of the Bruch's layer (B) establishing usherin as a basement membrane protein. Note the proximity of the type I collagen fibrils with the basement membrane in B. CL=capillary lumen; MC=marginal cell; IPM=interphotoreceptor cell matrix; BL=Bruch's layer. Magnification bars are 50 μtm.

FIG. 8 is a Western blot illustrating the direct interaction of usherin with type IV collagen and the indirect interaction of usherin with type I collagen. The LE domain of usherin interacts with type IV collagen (panels A and B). Extracts of matrix from the indicated mouse tissues were (A) reacted with the fusion peptide comprising the LE-domain, immunoprecipitated with anti-GST antibodies, and the immunoprecipitate western blotted using anti-type IV collagen antibodies, or (B) directly immunoprecipitated with anti-type IV collagen antibodies and the immunoprecipitate western blotted using anti-usherin antibodies. The molecular weight markers are given in kilodaltons. The LN domain of usherin interacts with type I collagen (panel C). Extracts from the indicated tissues were reacted with the fusion peptide comprising the LN domain and immunoprecipitated with anti-GST antibodies. The immunoprecipitate was analyzed by western blot and probed with antibodies specific for type I collagen.

FIG. 9 is a Western blot illustrating the interaction of usherin with itself, possibly forming a suprastructural network integrated into the basement membrane architecture. In panel A, the indicated fusion peptides were mixed with protein extracts from the eye, after removal of the lens lanes 1, 3, 4, 6, 7, and 9 or the liver (lanes 2, 5, and 8) or with pre-immune serum (lanes 3, 6, and 9). The immunoprecipitate was analyzed by western blot probed with anti-usherin antibodies. Only the LN domain was capable of immunoprecipitating usherin from retinal extracts (lane 1). In panel B, purified fusion peptides were mixed in various combinations and crosslinked using dimethyl superimidate (crosslinked mixtures are followed by an “X”). Products were resolved by PAGE, and stained with Coomassie blue. Arrows denote dimeric and trimeric crosslinked product.

### DEFINITIONS

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

For convenience, the meaning of certain terms and phrases employed in the specification, examples, and appended claims are provided below.

Unless otherwise specified, “a”, “an,” “the,” and “at least one” as used herein, are used interchangeably and mean one or more than one. Thus, for example, reference to “an antibody” includes a mixture of two or more antibodies.

The term “assay” or “immunoassay,” as used herein, is meant to refer to an assay method, such as enzyme immunoassay, enzyme-linked immunosorbent assay, immunodiagnostic, a radio-immunoassay, and the like, that uses antibodies (monoclonal or polyclonal) to detect and quantify a polypeptide, such as human usherin protein.

The terms “biological sample” refer to a sample of tissue or fluid isolated from an individual, including but not limited to, for example, at least a portion of testis, cochlea, epididymus, ovary, eye, uterus, heart, pancreas, prostate, skin, placenta, spleen, submaxillary gland, small intestine, large intestine, blood vessels, and combinations thereof.

The term “complement” or “complementary,” as used herein, is meant to refer to the ability of two single stranded polynucleotides to base pair with each other, where an adenine on one polynucleotide will base pair to a thymine on a second polynucleotide and a cytosine on one polynucleotide will base pair to a guanine on a second polynucleotide. Two polynucleotides are complementary to each other when a nucleotide sequence of one polynucleotide can base pair with a nucleotide sequence in the second polynucleotide. For instance, 5′-ATGC and 5′-GCAT are complementary. The term complement and complementary also encompasses two polynucleotides where one polynuceotide contains at least one nucleotide that will not base pair to at least one nucleotide present on a second polynucleotide. For instance, the third nucleotide of each of the two polynucleotides 5′-ATTGC and 5′-GCTAT will not base pair, but these two polynucleotides are complementary as defined herein.

The term “epitope” or “epitopic,” as used herein, refers to the site on an antigen or hapten to which specific B cells and/or T cells respond. The term is also used interchangeably with “antigenic determinant” or “antigenic determinant site.” Antibodies that recognize the same eptiope can be identified in a simple immunoassay showing the ability of one antibody to block the binding of another antibody to a target antigen.

The phrase “highly stringent hybridization conditions,” as used herein, is meant to refer to conditions such as 6× SSC, 5× Denhardt, 0.5% Sodium Dodecyl Sulfate (“SDS”), and 100 micrograms per milliliter (“μg/ml”) fragmented and denatured salmon sperm DNA hybridized overnight at 65° C. and washed in 2× SSC, 0.1% SDS one time at room temperature for about 10 minutes followed by one time at 65° C. for about 15 minutes followed by at least one wash in 0.2× SSC, 0.1% SDS at room temperature for at least 3-5 minutes.

The term “immunoreact,” “immunoreacts,” or “immunoreactive,” as used herein, refers to the ability of an antibody, monoclonal or polyclonal, to recognize and specifically bind to an antigen. Thus, for example, an antibody is immunoreactive with a human usherin protein when the antibody recognizes and binds to a specific epitope or site contained within the polypeptide and forms an immunoconjugate. The term “immunoconjugate” or “immunoconjugates,” as used herein, is meant to refer to an antibody/antigen complex formed when the antibody immunoreacts with the antigen, e.g., protein complex formed when an antibody immunoreacts with an usherin protein.

Immunoreactivity may be determined by antibody binding, more particularly, by the kinetics of antibody binding, and/or by competition in binding using as competitor(s) a known polypeptide(s) containing an epitope against which the antibody is directed. The techniques for determining whether a polypeptide is immunoreactive with an antibody are known in the art.

The term “polynucleotide” refers to a polymeric form of nucleotides of any length, either ribonucleotides or deoxyribonucleotides, and includes both double- and single-stranded DNA and RNA. A polynucleotide may include nucleotide sequences having different functions, including for instance coding sequences and non-coding sequences such as regulatory sequences. A polynucleotide can be obtained directly from a natural source, or can be prepared with the aid of recombinant, enzymatic, or chemical techniques. A polynucleotide can be linear or circular in topology. A polynucleotide can be, for example, a portion of “USH2A,” which is DNA sequence, as shown in Table 2 (SEQ ID NO:3), GenBank Accession No. AF055580, encoding for the USH2a protein.

The term “polypeptide” as used herein refers to a polymer of amino acids and does not refer to a specific length of a polymer of amino acids. Thus, for example, the terms peptide, oligopeptide, protein, and enzyme are included within the definition of polypeptide. This term also includes post-expression modifications of the polypeptide, for example, glycosylations, acetylations, phosphorylations, and the like. An “usherin protein” or “USH2a protein” or “usherin” or “USH2A diagnostic protein,” as used herein, refers to a polypeptide that is expressed by an individual, by a coding region isolated from an individual, by a coding region that hybridizes with a nucleotide sequence as described in greater detail herein, or by a coding region that has a certain percentage structural similarity with a nucleotide sequence. An usherin protein can be produced using recombinant techniques, or chemically or enzymatically synthesized. A coding region refers to a polynucleotide that encodes a polypeptide, usually via mRNA, when place under the control of appropriate regulatory sequences. The boundaries of the coding region are generally determined by a translation start codon at its 5′ end and a translation stop codon at its 3′ end.

### DETAILED DESCRIPTION OF PREFERRED EMBODIMENTS

The present invention provides methods and test kits for diagnosing individuals that are homozygous for mutations in the USH2A gene (SEQ ID NO:3, GenBank Accession No. AF055580) that encodes an usherin protein (SEQ ID NO:4). The usherin protein encoded by the USH2A gene has important structural and functional properties, since in its absence, people suffer congenital high frequency-specific sensorineural hearing loss and progressive retinitis pigmentosa, which are the defining pathologies of Usher syndrome Type IIa.

Conceptual translation of the USH2A gene (Table 2) open reading frame results in a protein consisting of 1551 amino acid residues (Table 3) with a predicted molecular weight of 171.5 kilodaltons and an isoelectric point of 7.45. A NCBI RPS-BLAST CD search of Genbank with the deduced USH2a protein sequence revealed a high degree of homology in the region from amino acid residues 300 to 1050 to all the laminin family members (32% identity and 47% similarity).

The polypeptide chain contains 10 laminin-type EGF-like domains (LE domains), each containing approximately 50 amino acid residues, arranged in tandem. The laminins are one of the major components forming the extracellular matrix of basement membranes in all tissues and the LE motif is present in other extracellular matrix proteins. Homology between the USH2a protein and the laminins ends at position 1050, however, and an analysis of the carboxy terminal region from 1050 to 1551 using the Paracoil program (MIT) did not identify the characteristic coiled-coil domains present in all laminins identified thus far. From position 1090 to 1500, however, the USH2a protein has four homologous tandem repeats of approximately 100 residues homologous to a variety of proteins containing fibronectin type-III (F3) repeats. The first 20 residues of the USH2a protein are highly hydrophobic with characteristics of a signal peptide and may represent a signal for secretion. In addition, the protein contains 18 potential n-glycosylation sites, and the KQEL endoplasmic reticulum (ER) targeting sequence is present at position 1429.

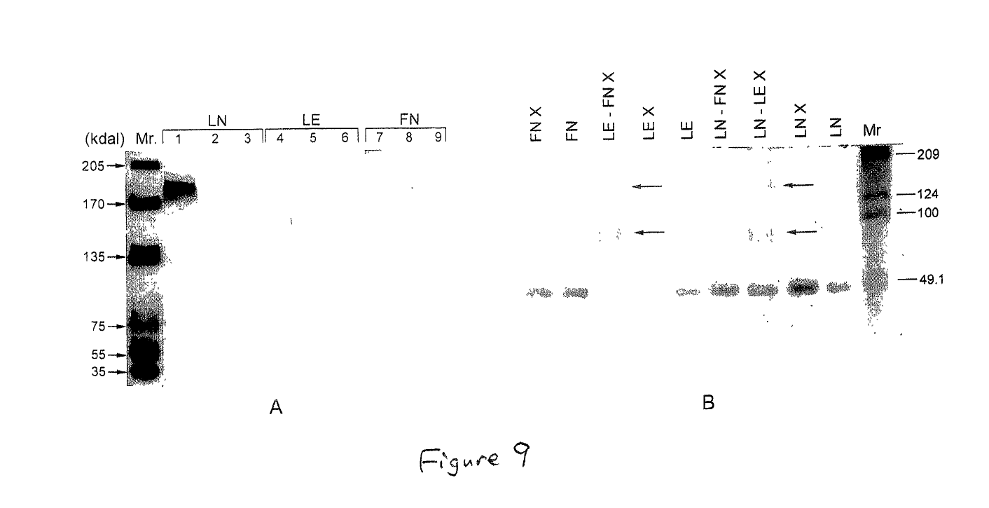

The USH2A gene encodes a novel protein with three main structural motifs. On the amino terminus is an LN module. This globular domain is a common feature of laminins, found in six of the known chains (α1, α2, α5, β1, and β2), where, like usherin, they are followed by the rod-like laminin-EGF-like modules (LE domains) (Bork et al, Q. Rev. Biophys, 29:119-167 (1996); Beck et al., FASEB J., 4:148-160 (1990)). These domains are required for the polymerization of laminins into the characteristic networks found in basement membranes (Bruch et al., Eur. J. Biochem, 185:271-279 (1989); Yurchenco et al., J. Biol. Chem., 268:17286-17299 (1993)). The LN domain from laminin α1 chain has been studied extensively, and found to bind specifically integrins α1β1 and α2β1, and to the heparin sulfate domains of perlecan (Pfaff et al., Eur. J. Biochem., 225:975-984 (1994); Colognato-Pyke et al., J. Biol. Chem., 270:9398-9406 (1995); Ettner et al., FEBS Lett., 430:217-221 (1998)). The LN domain of the usherin protein is functionally significant. It may be important for usherin network assembly, as suggested by the role of this domain in usherin-usherin interactions (FIG. 9). The LN domain also may function as a ligand for the cell surface receptors, such as the integrins. The absence of usherin results in developmental defects in the inner ear and progressive retinal pathology, which would be consistent with disruption of signaling processes required for normal cellular homeostasis.

The LN module of usherin has the most homology with that of netrin-1 (44% amino acid sequence identity for human). Netrin-1 is viewed as an axonal chemoattractant matrix molecule that plays a role in the guidance of efferent nerve fibers (Leonardo et al., Cold Spring Harb. Symp. Quant. Biol., 62:467-478 (1997); Metin et al., Development, 124:5063-5074 (1997)). Interestingly, netrin-1 plays a role in axon guidance of the optic nerve (Deiner et al., Neuron, 19:575-589 (1997)) as well as axon outgrowth from the cochlear nucleus in the brain (Poe et al., Brain Res. Dev. Brain Res., 105:153-157 (1998)). The similarity between usherin and netrin at both the structural and, potentially, the functional levels suggests that these comparisons should be considered as the research into usherin function proceeds.

The LE domain is comprised of repeat units of 60 amino acids containing 8 conserved cysteines (Engel, FEBS Lett., 251:1-7 (1989)). All of the known laminin chains, as well as some other extracellular matrix molecules including the netrins, contain multiple copies of this structural element, where the domain is present in 3 to 22 consecutive copies. The array of LE domains form rod-like tertiary structures with low flexibility (Beck et al., FASEB J., 4:148-160 (1990)). The LE domain of the murine laminin gamma-I chain has been shown to bind to nidogen, which is an important structural protein found in basement membranes (Mayer et al., FEBS Lett., 365:129-132 (1995)). The usherin protein contains 10 repeat units in its LE domain, and, as for the laminins, it is believed that this domain likely plays more of a structural than a functional role, e.g., by providing a rigid spacer between the two functional domains of the molecule. Provided is strong evidence that the LE domain interacts with type IV collagen. The fact that anti-collagen (IV) antibodies immunoprecipitate the complex from tissue extracts suggests that the interaction is of high affinity, and illustrates that the interaction does indeed occur between native usherin and type IV collagen. The usherin-collagen (W) interaction may serve to physically integrate collagen and usherin networks.

At the carboxy terminus of the usherin protein are three fibronectin Type III repeats. These elements are approximately 100 amino acids in length and are a shared domain with at least 45 different families of molecules ranging from cytokine receptors to cell surface binding proteins. The domain is not conserved at the amino acid level, but rather its structural motif where different Type III domains may be almost completely dissimilar at the amino acid level and as much as 90% structurally similar (Sharma et al., EMBO J., 18: 1468-1479 (1999)). Like the LE domains, the fibronectin Type III domains tend to be present in a tandem series of variable length, forming a series of beta-pleated sheet structures. They are known to function as heparin binding molecules (Barkalow et al., J. Biol. Chem., 266:7812-7818 (1991)) as well as integrin binding molecules (Bowditch et al., J. Biol. Chem., 269:10856-10863 (1994)). Recent evidence demonstrates multimerization of fibronectin type III domains in the recruitment of a variety of integrin heterodimers (Silletti et al., J. Cell Biol., 149(7):1485-1502 (Jun. 26, 2000)).

Review of the canonical domains of the usherin protein suggest two functional domains are linked by a rigid rod-like structural domain comprised of LE repeats. These LE repeats act as a scaffold for type IV collagen interaction as well usherin-usherin interaction, facilitating the potential formation of usherin networks that are physically integrated into the basement membrane architecture. The LN and fibronectin type III domains may have multiple functions, playing roles in structural integration of the usherin network as well as interacting with cell surface receptors to modulate tissue homeostasis. The role of usherin in maintaining tissue homeostasis may not be important (or redundant pathways for its function may exist) in most of the tissues where it is expressed, however in the basement membranes of the retina and the inner ear usherin is required for normal development and homeostasis.

The methods and test kits of the present invention allow one of skill in the art to detect the presence or absence and also the concentration, if desired, of an usherin protein encoded by the USH2A gene in the sample, as well as other polypeptides, wherein a complement of a polynucleotide encoding the usherin protein is capable of hybridizing to the polynucleotide represented by SEQ ID NO:3 under highly stringent hybridization conditions.

There will likely be some percentage of individuals with Usher syndrome Type IIa that continue to express immunoreactive usherin in their tissues. Although the inventor does not intent to be bound by any particular theory or mechanism, it is believed that some of these people may possess a functionally inactive usherin protein, however, the protein would still be detectable in the basement membranes. These people would most likely carry missense mutations, which are relatively rare in the Usher syndrome Type IIa population based on mutation screening in currently available families (Weston et al., Am. J. Hum. Genet., 66:1199-1210 (2000)). Alternatively, it is envisioned that the immunoreactive usherin protein could be functional, and that the individual is exhibiting the Usher type IIa syndrome phenotype because of a mutation in the 5′ untranslated region of the USH2A gene, e.g., promoter region, which causes the usherin protein production levels to be downregulated. In this latter scenario, the usherin protein is a functioning usherin protein, however, the Usher type Ia syndrome results because it is not present at high enough levels to maintain normal tissue function.

On the other hand, nonsense and frame-shift mutations, as well as insertions and deletions will likely result in the complete absence of usherin protein in the basement membranes. As an example of the expected frequency of detection using this approach is its application for diagnosis of X-linked Alport syndrome, where immunoscreening of skin biopsy is able to predict genetic pre-disposition in about 70% of the cases examined (van der Loop et al., Kidney Int., 55:1217-1224 (1999)).

The methods and test kits of the present invention entail the acquisition of tissue that normally express the usherin protein if present in a subject. Usherin can normally be found in both capillary and structural basement membranes from only certain bodily organs, such as the retina, inner ear, spleen, testis, ovary, epididymus, submaxillary gland, large and small intestine. The biological sample of the present invention may be obtained from tissue selected from the group consisting of testis, cochlea, epididymus, ovary, eye, uterus, heart, pancreas, prostate, skin, placenta, spleen, submaxillary gland, small intestine, large intestine, blood vessels, and combinations thereof. The placenta is an example of a preferred tissue because it can be obtained non-invasively following birth, however, any tissue that can be obtained with minimum risk to the patient, in which the usherin protein is normally expressed, would be equally suitable. Many organs, however, are completely devoid of usherin, including the brain, skin, kidney, lung, liver, skeletal muscle, and smooth muscle.

The acquired tissue would be fixed by immersion in any suitable fixative that does not affect the reactivity of the usherin protein with the antibody preparation. Both phosphate buffered formalin, as is commercially available to pathologists from a variety of sources, and phosphate buffered paraformaldehyde (4% w/vol) are examples of suitable fixatives. The fixed tissue is embedded in paraffin wax using standard embedding procedures known to the art and sections cut with a paraffin microtome.

The methods of the present invention also provide for the use of antibodies that are immunoreactive with an usherin protein encoded by the USH2A gene, as well as other polypeptides, wherein a complement of a polynucleotide encoding the usherin protein is capable of hybridizing to the polynucleotide represented by SEQ ID NO:3 under highly stringent hybridization conditions. Preferably, the antibodies selectively recognize the usherin protein epitopes and bind to these epitopes with high affinity. The antibodies can be used multiply to bind to different usherin epitopes such as in sandwich assays. These antibodies can have substances that act as labels attached to them for ease of identification following binding of the antibody to the usherin protein, if present in the sample. The antibodies of this invention bind to the usherin protein with specificity so that epitopes of the usherin protein can be detected with particularity in a biological sample.

Antibodies which can be used in accordance with the present invention are antibodies that are reactive with the USH2a protein or other polypeptides in which the complement of a polynucleotide encoding the usherin protein is capable of hybridizing to the polynucleotide represented by SEQ ID NO:3 under highly stringent hybridization conditions. An antibody encompassed by the present invention is an antibody that can immunoreact with any portion of the usherin protein. Preferably, an antibody of the present invention immunoreacts with the LN domain of the usherin protein (SEQ ID NO:2) and/or with SEQ ID NO:1. The term antibody is also intended to encompass both polyclonal and monoclonal antibodies. The term antibody is intended to encompass mixtures of more than one antibody reactive with the usherin protein (e.g., a cocktail of different types of monoclonal and/or polyclonal antibodies reactive with the usherin protein). The term antibody is further intended to encompass whole antibodies, biologically functional fragments thereof, single chains or single chain fragments with usherin protein binding properties, and chimeric antibodies including portions from more than one species, bifunctional antibodies, etc. Biologically functional antibody fragments which can be used are those fragments sufficient for binding of the antibody fragment to usherin protein.

The chimeric antibodies can comprise portions derived from two different species (e.g., human constant region and murine variable or binding region). The portions derived from two different species can be joined together chemically by conventional techniques or can be prepared as fusion proteins using genetic engineering techniques well known in the art. In addition, DNA encoding the proteins of both the light chain and heavy chain portions of the chimeric antibody can be expressed together as fusion proteins.

The antibodies of the present invention preferably are selected so as not to cross-react with other cellular components that are contained within the biological sample. The antibodies can be of any class and subclass determined by the Ouchterlony double diffusion test. Antibodies of the IgG class are preferred. Alternatively, antibodies which recognize usherin protein can be synthesized by biosynthetic or recombinant means, either in whole or in part.

In addition, the antibodies can be labeled with a variety of detectable molecules known in the art, including radioactive and nonradioactive labels. Typical radioactive labels include ³²P, ³³P, ³⁵S, ¹²⁵I, and the like. Non-radioactive labels include, for example, ligands such as biotin or digoxigenin as well as enzymes such as phosphatase or peroxidases, or the various chemiluminescers such as luciferin, or fluorescent compounds like fluorescein and its derivatives, bioluminescent compounds, and other labels known to one of skill in the art.

A variety of techniques are known and available to an artisan in assaying for the presence or absence of an antigen, such as usherin protein, and its concentration level, if desired, within in a biological sample. These immunoassays are quick and accurate tests that can be used on-site and in the laboratory to detect specific molecules. Immunoassays rely on the inherent ability of an antibody to bind to the specific structure of a molecule. Preferably, the antibodies of the present invention are highly specific for and will only bind to an usherin protein. Such assays include, but are not limited to, Western blots; agglutination test; enzyme-labeled and mediated immunoassays, such as Enzyme Linked Immunosorbent Assays (ELISA); biotin/avidin type assays; radioimmunoasssays; immunoelectrophoresis; immunoprecipitation, etc. The reactions generally include revealing labels such as fluorescent, chemiluminescent, bioluminescent, radioactive, enzymatic labels or dye molecules, or other methods for detecting the formation of a complex between the antigen and the antibody or antibodies reacted therewith.

Conventionally, various methods for detecting and/or measuring antigen concentration have been known, some of which are used for clinical diagnosis. Of these methods for measuring antigen concentration, the one commonly called sandwich Enzyme Linked Immunosorbent Assay (ELISA) method (or sandwich radio-immunoassay (RIA) method) is a well-known and widely used immunoassay in the art. This method is characterized by determining the presence or absence of and measuring the concentration of an antigen (e.g., usherin protein) using two kinds of monoclonal antibodies which recognize different epitopes of the antigen, or alternately, with one kind of monoclonal antibody and one kind of polyclonal antibody. The antigen of the present invention that is to be detected in the immunoassays are an usherin protein as well as other polypeptides encoded by a polynucleotide encoding the usherin protein wherein the complement thereof is capable of hybridizing to the polynucleotide represented by SEQ ID NO:3 under highly stringent hybridization conditions.

The procedure of this sandwich ELISA consists of three stages. In the first stage, a biological sample is poured on a measurement plate on which monoclonal/polyclonal antibodies (primary antibodies) have been absorbed; the usherin protein, if present in the biological sample, is bound to the primary antibodies. In the second stage, the substances in the biological sample other than the usherin protein are washed off with a washing agent. Then, in the third stage, a solution of the secondary antibodies, labeled with reporter molecules, such as an enzyme, radioisotope, and the like, are poured on the plate; the labeled antibodies bind to the usherin protein having been bound to the primary antibodies. Excessive labeled antibodies are fully rinsed away with washing agent, then the amount of the reporter molecules left in the measurement plate is measured by means of an enzyme activity reader or a liquid scintillation counter; and the observed values are used for the estimation of the quantity of the usherin protein in the biological sample. The presence of usherin protein in biological tissue that normally expresses the usherin protein is indicative of an individual not having Usher syndrome Type IIa. On the other hand, the absence of usherin protein in biological tissue that normally expresses the usherin protein is indicative of an individual having or being at risk for developing Usher syndrome Type Ila.

Another immunoassay well-known in the art in determining the presence or absence of an antigen and measuring its concentration is the competitive inhibition immunoassay. Generally, this method is often used to measure small antigens because competitive inhibition assays only require the binding of one antibody, rather than two as used in the standard sandwich formats as described above. Because of the high probability for steric hindrance occurring when two antibodies attempt to bind to a small molecule at the same time, a sandwich assay format may not be feasible, therefore a competitive inhibition assay would be preferable under these circumstances. The USH2a protein, however, has a predicted molecular weight of 171.5 kilodaltons and is large enough to bind two antibodies. The competitive inhibition immunoassay procedure is encompassed by the present invention to detect and/or measure usherin protein.

In this one antibody immunoassay, a monoclonal or polyclonal antibody is coated onto a 96-well microtiter plate. Preferably, the antibody is a monoclonal antibody. The biological sample is then added prior to or simultaneously with labeled usherin protein. Both labeled usherin protein, which is provided in one embodiment of a test kit, and unlabeled usherin protein (evaluating for presence in biological sample), compete for the binding site on the attached monoclonal or polyclonal antibody on the plate. This means that the labeled usherin protein will not be bound by the attached antibody on the plate if the antibody has already bound unlabeled usherin protein from the sample. The amount of unlabeled usherin protein in the sample is inversely proportional to the signal generated by the labeled usherin protein. The usherin protein can be labeled with a detectable label, which includes radioactive and nonradioactive labels. Typical radioactive labels include ³²P, ³³P, ³⁵S, ¹²⁵I, and the like. Non-radioactive labels include, for example, ligands such as biotin or digoxigenin as well as enzymes such as phosphatase or peroxidases, or the various chemiluminescers such as luciferin, or fluorescent compounds like fluorescein and its derivatives, bioluminescent compounds, and other labels known to one of skill in the art. Preferably, the label is an enzymatic moiety.

An individual “has” Usher syndrome Type IIa when their usherin protein levels as determined by the disclosed immoassays are below normal and the individual is suffering from conditions associated with the syndrome, for instance, hearing loss and a progressive loss of vision. An individual is “at risk” for developing Usher syndrome Type IIa when their usherin protein levels as determined by the disclosed immoassays are below normal and the individual is not suffering from conditions associated with the syndrome.

The usefulness of these assays is readily apparent; a relatively simple assay that is predictive of the presence or absence of Usher syndrome Type IIa.

Test kits are also embodiments of this invention. These test kit components are provided in order to perform the immunoassays, such as a competitive inhibition assay or an ELISA assay as described above. The immunoassays are performed to determine whether an individual has or is at risk for developing Usher syndrome Type IIa. The presence of usherin protein is indicative of an individual not having Usher syndrome Type IIa, while the absence of usherin protein is indicative of an individual having or being at risk for developing Usher syndrome Type IIa.

In one embodiment the test kit contains at least one monoclonal or polyclonal antibody that immunoreacts with at least a portion of the usherin protein, and a detectably-labeled usherin protein (e.g., competitive inhibition assay).

Another embodiment of a test kit of the present invention contains a first monoclonal or polyclonal antibody that immunoreacts with a portion of the usherin protein, and optionally a second monoclonal or polyclonal antibody that immunoreacts with another portion of the usherin protein, which are needed to perform immunoassays, such as ELISA or RIA as described above, for the detection of usherin protein that may be present in a biological sample obtained from individuals (e.g., ELISA assay).

Optionally, the test kits may also contain the solid supports, such as microtiter trays, for performing the assays. Instructions for performing the assays for usherin protein can also be included in the kits. If desired, an identification label can be attached to an antibody of the test kits. In preferred embodiments of the test kits, antibodies are provided that allow sandwich assays to be performed. In particularly preferred embodiments of the invention, one of the sandwich antibodies is unlabeled and attached to a solid support. The other antibody has a label bound to it for detection purposes.

Objects and advantages of this invention are further illustrated by the following examples, but the particular materials and amounts thereof recited in these examples, as well as other conditions and details, should not be construed to unduly limit this invention.

### Sequence Free Text

SEQ ID NO:1—immunogen

SEQ ID NO:2—immunogen, amino acids 318 to 518 of USH2a protein

SEQ ID NO:3—USH2A gene, polynucleotide sequence encoding human usherin protein

SEQ ID NO:4—USH2a protein, polypeptide encoded by the USH2A gene

### EXAMPLE 1

### Identification of Tissue That Normally Expresses Usherin mRNA and Protein

Antibodies.

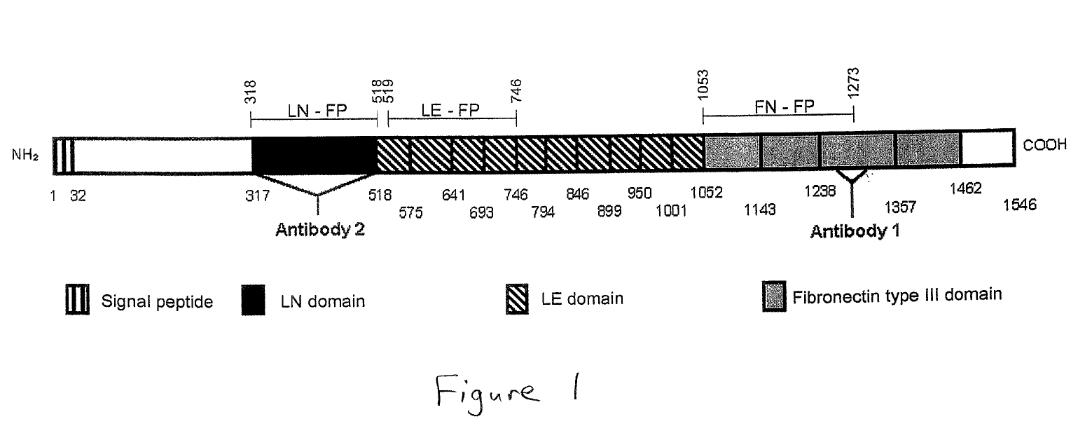

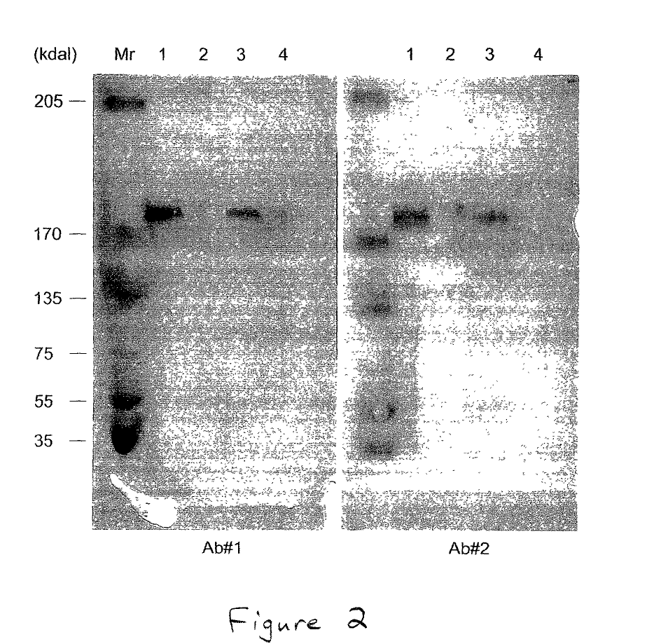

Antibodies were developed that are highly specific and useful for immunohistochemistry, immunoprecipitation, and western blotting. Antibody 1 was developed against a synthetic peptide corresponding to a 23 amino acid sequence in the murine exon 17 (towards the middle to carboxy-tenninal end of the usherin protein). The peptide sequence of the immunogen was: QAPPQTQGPPTVWKISPTELRIE which is represented by SEQ ID NO:1. Antibody 2 was developed against the entire LN domain of usherin (the immunogen is represented by SEQ ID NO:2 or amino acids 318 to 518, based on the translated cDNA)(see FIG. 1), which was expressed using the FLAG-ATS system (Sigma, St. Louis). The peptide sequence for SEQ ID NO:2 is shown in Table 1 below. Both antibodies were raised in rabbits and the reactive immunoglobulin was affinity purified using the immunogen. Specificity was verified by western blot of protein extracts from testis (which expresses usherin) and kidney (which does not express usherin). The antibody detects a single band of the appropriate molecular size of usherin (about 180 kilodaltons). With the exception of FIG. 2, the data presented is all derived through the use of antibody 2, however antibody 1 gave identical results.

TABLE 1
The polypeptide sequence represented by SEQ ID NO:2 (amino
acids 318-518 of the human usherin protein) is as follows:

```
Pro Leu Ala Gln Arg Tyr Cys Ile Pro Asn Asp Ala Gly Asp Thr Ala
1 5 10 15
Asp Asn Arg Val Ser Arg Leu Asn Pro Glu Ala His Pro Leu Ser Phe
                        20 25 30
Val Asn Asp Asn Asp Val Gly Thr Ser Trp Val Ser Asn Val Phe Thr
                35 40 45
Asn Ile Thr Gln Leu Asn Gln Gly Val Thr Ile Ser Val Asp Leu Glu
        50 55 60
Asn Gly Gln Tyr Gln Val Phe Tyr Ile Ile Ile Gln Phe Phe Ser Pro
65 70 75 80
Gln Pro Thr Glu Ile Arg Ile Gln Arg Lys Lys Glu Asn Ser Leu Asp
                                85 90 95
Trp Glu Asp Trp Gln Tyr Phe Ala Arg Asn Cys Gly Ala Phe Gly Met
                        100 105 110
Lys Asn Asn Gly Asp Leu Glu Lys Pro Asp Ser Val Asn Cys Leu Gln
                115 120 125
Leu Ser Asn Phe Thr Pro Tyr Ser Arg Gly Asn Val Thr Phe Ser Ile
        130 135 140
Leu Thr Pro Gly Pro Asn Tyr Arg Pro Gly Tyr Asn Asn Phe Tyr Asn
145 150 155 160
Thr Pro Ser Leu Gln Glu Ser Val Lys Ala Thr Gln Ile Arg Phe His
                                165 170 175
Phe His Gly Gln Tyr Tyr Thr Thr Glu Thr Ala Val Asn Leu Arg His
                        180 185 190
Arg Tyr Tyr Ala Val Asp Glu Ile Thr
                195 200
```

TABLE 2
The polynucleotide sequence represented by SEQ ID NO:3 encoding
for the human usherin protein, USH2a, is as follows:

tgtttgctct gcagaatact ttacctgggc accaagtctt ccttccagca ttcctgctgc
60
tacagcctat ttgctgagta accaggggtt acagcagcgt tgccaggcaa cgagggacag
120
cggtcctgtt gaagagccat ttgtcacact gaggggactg gttgaaatgc aataaagaaa
180
tgataccagc agctactcat gtcttcgcca ttgctaagaa cgtcgttggt attaccttac
240
tctgagaacg tgtctgcagt ttccagaaaa tggagtatcg caacatcact taaagtaccc
300
tgcttcaaag tattgctggc aagtggcgtg ggcctgatta tttatttaga aatgctttat
360
caggaggaga atgctttttg taaacatgaa ttgcccagtt ctttcattgg gctctggctt
420
cttgtttcag gtcattgaaa tgttgatctt tgcctatttt gcttcaatat ccttgactga
480
gtcacgaggt cttttcccaa ggctggagaa cgtgggagct ttcaagaaag tttccatcgt
540
gccaacccaa gcagtatgtg gactcccaga ccgaagcact ttttgtcaca gctctgctgc
600
tgctgaaagt attcagttct gtacccagcg gttttgtatt caggattgcc catacagatc
660
ttcacaccct acctacactg cccttttctc agcaggcctc agtagctgca tcacaccaga
720
caagaatgat ctgcatccta acgcccatag caattctgca agttttattt ttggaaatca
780
caagagctgc ttttcttctc ctccttctcc aaagctgatg gcatcattta ccttagctgt
840
atggctgaaa cctgagcaac aaggtgtaat gtgtgttata gaaaagacrg tagatgggca
900
gattgtgttc aaacttacaa tatctgagaa agagaccatg ttttattatc gcacagtaaa
960
tggtttgcaa cctccaataa aagtaatgac actggggaga attcttgtga agaaatggat
1020
tcatcttagt gtgcagqtgc atcagacaaa aatcagcttc tttatcaatg gcgtggagaa
1080
ggatcataca cctttcaatg caagaactct aagtggttca attacagatt ttgcatctgg
1140
tactgtgcaa ataggacaga gtttaaatgg tttagagcag tttgtcggaa gaatgcaaga
1200
ttttcgatta taccaagtgg cacttacaaa cagagagatt ctggaagtct tctctggaga
1260
tcttctcaga ttgcatgccc aatcacattg ccgttgccct ggcagccacc cgcgggtcca
1320
ccctttggca cagcggtact gcattcctaa tgatgcagga gacacagctg ataatagagt
1380
gtcacggttg aatcctgaag cccatcctct ctcttttgtc aatgataatg atgttggtac
1440
ttcatgggtt tcaaatgtgt ttacaaacat tacacagctt aatcaaggag tgactatttc
1500
agttgatttg gaaaatggac agtatcaggt gttttatatt atcattcagt tctttagtcc
1560
acaaccaacg gaaataagga ttcaaaggaa gaaggaaaat agtttagatt gggaggactg
1620
gcaatatttt gccaggaatt gtggtgcttt tggaatgaaa aacaatggag atttggaaaa
1680
acctgattct gtcaactgtc ttcagctttc caattttact ccatattccc gtggcaatgt
1740
cacatttagc atcctgacac ctggaccaaa ttatcgtcct ggatacaata acttctataa
1800
taccccatct cttcaagagt ccgtaaaagc cacgcaaata aggtttcatt ttcatgggca
1860
gtactataca actgagactg ctgttaacct cagacacaga tattatgcag tggacgaaat
1920
caccattagt gggagatgtc agtgccatgg tcatgccgat aactgcgaca caacaagcca
1980
gccatataga tgcctctgct cccaggagag cttcactgaa ggacttcatt gtgatcgctg
2040
cttgcctctt tataatgaca agcctttccg ccaaggtgat caagtttacg ctttcaattg
2100
taaaccttgt caatgcaaca gccattccaa aagctgccat tacaacatct ctgtagaccc
2160
atttcctttt gagcacttca gagggggagg aggagtttgt gatgattgtg agcataacac
2220
tacaqgaagg aactgtgagc tgtgcaagga ttactttttc cgacaagttg gtgcagatcc
2280
ttcggccata gatgtttgca aaccctgtga ctgtgataca gttggcacta gaaatggtag
2340
cattctttgt gatcagattg gaggacagtg taattgtaag agacacgtgt ctggcaggca
2400
gtgcaatcag tgccagaatg gattctacaa tctacaagag ttggatcctg atggctgcag
2460
tccctgtaac tgcaatacct ctgggacagt ggatggagat attacctgtc accaaaattc
2520
aggccagtgc aagtgcaaag caaacgttat tgggcttagg tgtgatcatt gcaattttgg
2580
atttaaattt ctccgaagct ttaatgatgt tggatgtgag ccctgccagt gtaacctcca
2640
tggctcagtg aacaaattct gcaatcctca ctctgggcag tgtgagtgca aaaaagaagc
2700
caaaggactt cagtgtgaca cctgcagaga aaacttttat gggttagatg tcaccaattg
2760
taaggcctgt gactgtgaca cagctggatc cctccctggg actgtctgta atgctaagac
2820
agggcagtgc atctgcaagc ccaatgttga agggagacag tgcaataaat gtttggaggg
2880
aaacttctac ctacggcaaa ataattcttt cctctgtctg ccttgcaact gtgataagac
2940
tgggacaata aatggctctc tgctgtgtaa caaatcaaca ggacaatgtc cttgcaaatt
3000
aggggtaaca ggtcttcgct gtaatcagtg tgagcctcac aggtacaatt tgaccattga
3060
caattttcaa cactgccaga tgtgtgagtg tgattccttg gggacattac ctgggaccat
3120
ttgtgaccca atcagtggcc agtgcctgtg tgtgcctaat cgtcaaggaa gaaggtgtaa
3180
tcagtgtcaa ccaggttttt atatttctcc aggcaatgcc actggctgcc tgccatgctc
3240
atgccataca actggcgcag ttaatcacat ctgtaatagc ctgactggtc agtgtgtttg
3300
ccaagatgct tccattgctg ggcaacgttg tgaccaatgc aaagaccatt actttggatt
3360
tgatcctcag actggaagat gtcagccttg taattgtcat ctctcaggag ccttgaatga
3420
aacctgtcac ttggtcacag gccagtgttt ctgtaaacaa tttgtcactg gctcaaagtg
3480
tgatgcttgt gttcccagtg caagccactt ggatgtcaac aatctattgg gttgcagcaa
3540
aactccattc cagcaacctc cgcccagagg acaagttcaa agttcttctg ctatcaatct
3600
ctcctggagt ccacctgatt ctccaaatgc ccactggctt acttacagtt tactcaggga
3660
tggttttgaa atctacacaa cagaggatca atacccatac agtattcaat acttcttaga
3720
cacagacctg ttaccatata ccaaatattc ctattacatt gagaccacca atgtgcatgg
3780
ttcaacaagg agtgtagctg tcacttacaa gacaaaacca ggggtcccag agggaaactt
3840
gactttaagt tatatcattc ctattggctc agactctgtg acacttacct ggacaacact
3900
ctcaaatcaa tctggtccca tagagaaata tattttgtcc tgtgcccctt tggctggtgg
3960
tcagccatgt gtttcctacg aaggtcatga aacctcagct accatctgga atctggttcc
4020
atttgccaag tacgattttt ctgtacaggc gtgtactagc gggggctgtt tacacagctt
4080
gcccattaca gtgaccacag cccaggcccc tccccaaaga ctaagtccac ctaagatgca
4140
gaaaatcagt tctacagaac ttcatgtaga atggtctcca ccagcggaac taaatggaat
4200
aattataaga tatgaactat acatgagaag actgagatct actaaagaaa ccacatctga
4260
ggaaagtcga gtttttcaga gcagtggttg gctcagtcct cattcatttg tagaatcggc
4320
caatgaaaat gcattaaaac ctcctcaaac aatgacaacc atcactggct tggagccata
4380
caccaagtat gagttcagag tcttagctgt gaatatggct ggaagtgtgt cttctgcctg
4440
ggtctcagaa agaacgggag aatcagcacc tgtattcatg atccctcctt cagtctttcc
4500
cctctcttcg tactctctca atatctcctg ggagaagcca gcagataatg ttacaagagg
4560
aaaagttgtg gggtatgaca tcaatatgct ttctgaacaa tcacctcaac agtctattcc
4620
catggcgttt tcacagctgt tgcacactgc taaatcccaa gaactatctt acactgtaga
4680
aggactgaaa ccttatagga tatatgagtt tactattact ctctgcaatt cagttggttg
4740
tgtgaccagt gcttcgggag caggacaaac tttagcagca gcaccagcac aactgaggcc
4800
acctctggtt aaaggaatca acagcacaac aatccatctt aagtggtttc cacctgaaga
4860
actgaatgga ccctctccta tatatcagct ggaaaggaga gagtcatctc taccagctct
4920
gatgaccacg atgatgaaag gaatccgttt cataggaaat gggtattgta aatttcccag
4980
ctccactcac ccagtcaata cagacttcac tggtaagtgt gtttgacatt gctttattta
5040
ggagacacga agctccaaaa tgttttctat attttcatat ccctttacaa tgaattttta
5100
ttatacctac ttagagaaat actaattcag ccctttgata gcttttgcct gattgtttca
5160
gcatgtccat ctttttagaa ttctggggaa aaaagtcagg taagtgaagg aaaggaaaaa
5220
taaaagatga agatgaagaa gcagccttat tggatcaaag tatgtgcttt gtatttgtct
5280
ttttgtgaag tatgtgccag gacatgtttc ttgaaatatt attcactgtg ttctctgagc
5340
aaatgagttt gcaaaatgcc ctcatgctat tggagattct cagtatgcac cccgttactg
5400
aaactccaaa aagcattgta agaaagctat tcaactttgc ttagctaatc atgcctaaca
5460
gatatttgat gtaatgtttt ctttttcttt ctcttgctgt ttccttcttc tttttttcac
5520
tgtgacaact taatatctca tgttctatga agaacattgt gggqaaaact aatcccaggg
5580
aaaagataac ttctctaagc caggactatg gtaaagcaag tgaggctctt gtttcggtca
5640
caaaatttaa aggcactaaa aaactcagtg ttaatgtaaa ttttaatgca atatttttaa
5700
aaatgaaaat caatgtgaaa gcactataaa aatattatca aaagcttaaa taaagacaga
5760
ttgaactctg taccagcaca atcctgcctc actggcctta ccctcctcct ggccttacta
5820
gtaccgcaat attttggaag tcccatgacc tctgtgactt acagcttcta atagcatgat
5880
ttcaatatag ctgtaaaaaa actctactta tggtacacca tttttccaat ttttaaaaaa
5940
atttacaaag tataagatat atattattat gtaaactcat aaagatgttc atttaatcat
6000
ccatgagaaa gtcattttgg agcaaatagc tagtctttaa aatattgcat atgtgaagac
6060
aatgaaatgg aattcgagct ataaaaattt gtattgtttt atttttactt aaaatagtaa
6120
atagtttgct tttcattgag actggctgct gatgcacctt ggtaatgaat catgattata
6180
ttctaactga gatatattga gattaatgca tgattaacta ctctctcagt acatcaaaat
6240
cattgcagag tattagaaat tgaaccattg agctaaaaat gctcaacttc tgctttatat
6300
tcttaaaatg gcaaaaaaaa aaaaaaaaaa
6330

TABLE 3

The amino acid sequence represented by SEQ ID NO:4 for the
human usherin protein, USH2a, is as follows:

```
Met Leu Phe Val Asn Met Asn Cys Pro Val Leu Ser Leu Gly Ser Gly
1 5 10 15
Phe Leu Phe Gln Val Ile Glu Met Leu Ile Phe Ala Tyr Phe Ala Ser
                        20 25 30
Ile Ser Leu Thr Glu Ser Arg Gly Leu Phe Pro Arg Leu Glu Asn Val
                35 40 45
Gly Ala Phe Lys Lys Val Ser Ile Val Pro Thr Gln Ala Val Cys Gly
        50 55 60
Leu Pro Asp Arg Ser Thr Phe Cys His Ser Ser Ala Ala Ala Glu Ser
65 70 75 80
Ile Gln Phe Cys Thr Gln Arg Phe Cys Ile Gln Asp Cys Pro Tyr Arg
                                85 90 95
Ser Ser His Pro Thr Tyr Thr Ala Leu Phe Ser Ala Gly Leu Ser Ser
                        100 105 110
Cys Ile Thr Pro Asp Lys Asn Asp Leu His Pro Asn Ala His Ser Asn
                115 120 125
Ser Ala Ser Phe Ile Phe Gly Asn His Lys Ser Cys Phe Ser Ser Pro
        130 135 140
Pro Ser Pro Lys Leu Met Ala Ser Phe Thr Leu Ala Val Trp Leu Lys
145 150 155 160
Pro Glu Gln Gln Gly Val Met Cys Val Ile Glu Lys Thr Val Asp Gly
                                165 170 175
Gln Ile Val Phe Lys Leu Thr Ile Ser Glu Lys Glu Thr Met Phe Tyr
                        180 185 190
Tyr Arg Thr Val Asn Gly Leu Gln Pro Pro Ile Lys Val Met Thr Leu
                195 200 205
Gly Arg Ile Leu Val Lys Lys Trp Ile His Leu Ser Val Gln Val His
        210 215 220
Gln Thr Lys Ile Ser Phe Phe Ile Asn Gly Val Glu Lys Asp His Thr
225 230 235 240
Pro Phe Asn Ala Arg Thr Leu Ser Gly Ser Ile Thr Asp Phe Ala Ser
                                245 250 255
Gly Thr Val Gln Ile Gly Gln Ser Leu Asn Gly Leu Glu Gln Phe Val
                        260 265 270
Gly Arg Met Gln Asp Phe Arg Leu Tyr Gln Val Ala Leu Thr Asn Arg
                275 280 285
Glu Ile Leu Glu Val Phe Ser Gly Asp Leu Leu Arg Leu His Ala Gln
        290 295 300
Ser His Cys Arg Cys Pro Gly Ser His Pro Arg Val His Pro Leu Ala
305 310 315 320
Gln Arg Tyr Cys Ile Pro Asn Asp Ala Gly Asp Thr Ala Asp Asn Arg
                                325 330 335
Val Ser Arg Leu Asn Pro Glu Ala His Pro Leu Ser Phe Val Asn Asp
                        340 345 350
Asn Asp Val Gly Thr Ser Trp Val Ser Asn Val Phe Thr Asn Ile Thr
                355 360 365
Gln Leu Asn Gln Gly Val Thr Ile Her Val Asp Leu Glu Asn Gly Gln
        370 375 380
Tyr Gln Val Phe Tyr Ile Ile Ile Gln Phe Phe Her Pro Gln Pro Thr
385 390 395 400
Glu Ile Arg Ile Gln Arg Lys Lys Glu Asn Ser Leu Asp Trp Glu Asp
                                405 410 415
Trp Gln Tyr Phe Ala Arg Asn Cys Gly Ala Phe Gly Met Lys Asn Asn
                        420 425 430
Gly Asp Leu Glu Lys Pro Asp Ser Val Asn Cys Leu Gln Leu Her Asn
                435 440 445
Phe Thr Pro Tyr Ser Arg Gly Asn Val Thr Phe Ser Ile Leu Thr Pro
        450 455 460
Gly Pro Asn Tyr Arg Pro Gly Tyr Asn Asn Phe Tyr Asn Thr Pro Ser
465 470 475 480
Leu Gln Glu Ser Val Lys Ala Thr Gln Ile Arg Phe His Phe His Gly
                                485 490 495
Gln Tyr Tyr Thr Thr Glu Thr Ala Val Asn Leu Arg His Arg Tyr Tyr
                        500 505 510
Ala Val Asp Glu Ile Thr Ile Ser Gly Arg Cys Gln Cys His Gly His
                515 520 525
Ala Asp Asn Cys Asp Thr Thr Ser Gln Pro Tyr Arg Cys Leu Cys Ser
        530 535 540
Gln Glu Her Phe Thr Glu Gly Leu His Cys Asp Arg Cys Leu Pro Leu
545 550 555 560
Tyr Asn Asp Lys Pro Phe Arg Gln Gly Asp Gln Val Tyr Ala Phe Asn
                                565 570 575
Cys Lys Pro Cys Gln Cys Asn Ser His Ser Lys Ser Cys His Tyr Asn
                        580 585 590
Ile Ser Val Asp Pro Phe Pro Phe Glu His Phe Arg Gly Gly Gly Gly
                595 600 605
Val Cys Asp Asp Cys Glu His Asn Thr Thr Gly Arg Asn Cys Glu Leu
        610 615 620
Cys Lys Asp Tyr Phe Phe Arg Gln Val Gly Ala Asp Pro Ser Ala Ile
625 630 635 640
Asp Val Cys Lys Pro Cys Asp Cys Asp Thr Val Gly Thr Arg Asn Gly
                                645 650 655
Ser Ile Leu Cys Asp Gln Ile Gly Gly Gln Cys Asn Cys Lys Arg His
                        660 665 670
Val Ser Gly Arg Gln Cys Asn Gln Cys Gln Asn Gly Phe Tyr Asn Leu
                675 680 685
Gln Glu Leu Asp Pro Asp Gly Cys Ser Pro Cys Asn Cys Asn Thr Ser
        690 695 700
Gly Thr Val Asp Gly Asp Ile Thr Cys His Gln Asn Ser Gly Gln Cys
705 710 715 720
Lys Cys Lys Ala Asn Val Ile Gly Leu Arg Cys Asp His Cys Asn Phe
                                725 730 735
Gly Phe Lys Phe Leu Arg Ser Phe Asn Asp Val Gly Cys Glu Pro Cys
                        740 745 750
Gln Cys Asn Leu His Gly Ser Val Asn Lys Phe Cys Asn Pro His Ser
                755 760 765
Gly Gln Cys Glu Cys Lys Lys Glu Ala Lys Gly Leu Gln Cys Asp Thr
        770 775 780
Cys Arg Glu Asn Phe Tyr Gly Leu Asp Val Thr Asn Cys Lys Ala Cys
785 790 795 800
Asp Cys Asp Thr Ala Gly Ser Leu Pro Gly Thr Val Cys Asn Ala Lys
                                805 810 815
Thr Gly Gln Cys Ile Cys Lys Pro Asn Val Glu Gly Arg Gln Cys Asn
                        820 825 830
Lys Cys Leu Glu Gly Asn Phe Tyr Leu Arg Gln Asn Asn Ser Phe Leu
                835 840 845
Cys Leu Pro Cys Asn Cys Asp Lys Thr Gly Thr Ile Asn Gly Ser Leu
        850 855 860
Leu Cys Asn Lys Ser Thr Gly Gln Cys Pro Cys Lys Leu Gly Val Thr
865 870 875 880
Gly Leu Arg Cys Asn Gln Cys Glu Pro His Arg Tyr Asn Leu Thr Ile
                                885 890 895
Asp Asn Phe Gln His Cys Gln Met Cys Glu Cys Asp Ser Leu Gly Thr
                        900 905 910
Leu Pro Gly Thr Ile Cys Asp Pro Ile Ser Gly Gln Cys Leu Cys Val
                915 920 925
Pro Asn Arg Gln Gly Arg Arg Cys Asn Gln Cys Gln Pro Gly Phe Tyr
        930 935 940
Ile Ser Pro Gly Asn Ala Thr Gly Cys Leu Pro Cys Ser Cys His Thr
945 950 955 960
Thr Gly Ala Val Asn His Ile Cys Asn Ser Leu Thr Gly Gln Cys Val
                                965 970 975
Cys Gln Asp Ala Ser Ile Ala Gly Gln Arg Cys Asp Gln Cys Lys Asp
                        980 985 990
His Tyr Phe Gly Phe Asp Pro Gln Thr Gly Arq Cys Gln Pro Cys Asn
                995 1000 1005
Cys His Leu Ser Gly Ala Leu Asn Glu Thr Cys His Leu Val Thr
        1010 1015 1020
Gly Gln Cys Phe Cys Lys Gln Phe Val Thr Gly Ser Lys Cys Asp
        1025 1030 1035
Ala Cys Val Pro Ser Ala Ser His Leu Asp Val Asn Asn Leu Leu
        1040 1045 1050
Gly Cys Ser Lys Thr Pro Phe Gln Gln Pro Pro Pro Arg Gly Gln
        1055 1060 1065
Val Gln Ser Ser Ser Ala Ile Asn Leu Ser Trp Ser Pro Pro Asp
        1070 1075 1080
Ser Pro Asn Ala His Trp Leu Thr Tyr Ser Leu Leu Arg Asp Gly
        1085 1090 1095
Phe Glu Ile Tyr Thr Thr Glu Asp Gln Tyr Pro Tyr Ser Ile Gln
        1100 1105 1110
Tyr Phe Leu Asp Thr Asp Leu Leu Pro Tyr Thr Lys Tyr Ser Tyr
        1115 1120 1125
Tyr Ile Glu Thr Thr Asn Val His Gly Ser Thr Arg Ser Val Ala
        1130 1135 1140
Val Thr Tyr Lys Thr Lys Pro Gly Val Pro Glu Gly Asn Leu Thr
        1145 1150 1155
Leu Ser Tyr Ile Ile Pro Ile Gly Ser Asp Ser Val Thr Leu Thr
        1160 1165 1170
Trp Thr Thr Leu Ser Asn Gln Ser Gly Pro Ile Glu Lys Tyr Ile
        1175 1180 1185
Leu Ser Cys Ala Pro Leu Ala Gly Gly Gln Pro Cys Val Ser Tyr
        1190 1195 1200
Glu Gly His Glu Thr Ser Ala Thr Ile Trp Asn Leu Val Pro Phe
        1205 1210 1215
Ala Lys Tyr Asp Phe Ser Val Gln Ala Cys Thr Ser Gly Gly Cys
        1220 1225 1230
Leu His Ser Leu Pro Ile Thr Val Thr Thr Ala Gln Ala Pro Pro
        1235 1240 1245
Gln Arg Leu Ser Pro Pro Lys Met Gln Lys Ile Ser Ser Thr Glu
        1250 1255 1260
Leu His Val Glu Trp Ser Pro Pro Ala Glu Leu Asn Gly Ile Ile
        1265 1270 1275
Ile Arg Tyr Glu Leu Tyr Met Arg Arg Leu Arg Ser Thr Lys Glu
        1280 1285 1290
Thr Thr Ser Glu Glu Ser Arg Val Phe Gln Ser Ser Gly Trp Leu
        1295 1300 1305
Ser Pro His Ser Phe Val Glu Ser Ala Asn Glu Asn Ala Leu Lys
        1310 1315 1320
Pro Pro Gln Thr Met Thr Thr Ile Thr Gly Leu Glu Pro Tyr Thr
        1325 1330 1335
Lys Tyr Glu Phe Arg Val Leu Ala Val Asn Met Ala Gly Ser Val
        1340 1345 1350
Ser Ser Ala Trp Val Ser Glu Arg Thr Gly Glu Ser Ala Pro Val
        1355 1360 1365
Phe Met Ile Pro Pro Ser Val Phe Pro Leu Ser Ser Tyr Ser Leu
        1370 1375 1380
Asn Ile Ser Trp Glu Lys Pro Ala Asp Asn Val Thr Arg Gly Lys
        1385 1390 1395
Val Val Gly Tyr Asp Ile Asn Met Leu Ser Glu Gln Ser Pro Gln
        1400 1405 1410
Gln Ser Ile Pro Met Ala Phe Ser Gln Leu Leu His Thr Ala Lys
        1415 1420 1425
Ser Gln Glu Leu Ser Tyr Thr Val Glu Gly Leu Lys Pro Tyr Arg
        1430 1435 1440
Ile Tyr Glu Phe Thr Ile Thr Leu Cys Asn Ser Val Gly Cys Val
        1445 1450 1455
Thr Ser Ala Ser Gly Ala Gly Gln Thr Leu Ala Ala Ala Pro Ala
        1460 1465 1470
Gln Leu Arg Pro Pro Leu Val Lys Gly Ile Asn Ser Thr Thr Ile
        1475 1480 1485
His Leu Lys Trp Phe Pro Pro Glu Glu Leu Asn Gly Pro Ser Pro
        1490 1495 1500
Ile Tyr Gln Leu Glu Arg Arg Glu Ser Ser Leu Pro Ala Leu Met
1505 1510 1515
Thr Thr Met Met Lys Gly Ile Arg Phe Ile Gly Asn Gly Tyr Cys
        1520 1525 1530
Lys Phe Pro Ser Ser Thr His Pro Val Asn Thr Asp Phe Thr Gly
        1535 1540 1545
Lys Cys Val
        1550
```

Immunoperoxidase Detection.

Immunoperoxidase detection was performed as described previously (Sayers et al, Kidney Int., 56:1662-1673 (1999)). Tissues were fixed by transcardial perfusion with 4% paraformaldehyde, removed, cut into pieces no larger than 2 millimeter (mm), and incubated in fixative for 2 hours at 5° C. before being embedded into paraffin blocks using standard embedding procedures. De-paraffined tissue sections were treated with 1% trypsin for 30 minutes in 5 millimolar (mM) Tris·Cl (pH 7.4) to expose hidden epitopes. The immunoperoxidase reaction was developed using the AEC kit (Vector Laboratories, Burlingame, Calif.). The type IV collagen antibody, used as a control for basement membrane staining, was purchased from Southern Biotechnology (Birmingham, Ala.). Tissues were taken from an adult (8 weeks) C57B 116 mice following trans-cardiac perfusion with phosphate buffer solution (PBS) followed by 4% paraformaldehyde in PBS. Slides were photographed, and the photo's were scanned using a Hewlett Packard Scanjet 4C/T, and assembled into montages using Adobe Photoshop. No sharpness or contrast enhancements were employed.

Immunogold Localization.

For ultrastructural localization of the usherin protein, a postembedding procedure was employed using Unicryl embedding media (Vector Laboratories, Burlingame, Calif.). Tissue was fixed by transcardiac perfusion of animals with PBS first followed by 4% paraformaldehyde. Tissues were removed, minced into 1 to 2 millimeter (mm) cubes, and post-fixed in 4% paraformaldehyde for 2 hours. The fixed tissue was then dehydrated by immersion through a series of graded ethanols (50-100%), and infiltrated with 100% resin. Infiltration was carried out by incubating for 1 hour on a shaker at room temperature for each of 2 changes, followed by a fresh change of resin and incubation overnight at room temperature. The next morning the tissue was embedded in flat polyethylene embedding molds and polymerized in an aluminum lined box using a 360 nanonmeter (nm) light positioned 10 centimeter (cm) from the specimen. Polymerization was complete after 36 hours at 4° C.

Blocks were cut at 70nm, and sections collected onto 200 mesh formvar/carbon coated grids (Electron Microscopic Sciences). The grids were floated on the surface of staining solutions. The primary antibodies were optimized by testing a series of concentrations. The optimal concentrations were about twice that for immunofluorescence detection. The primary antibody was added in a solution of blocking buffer containing 1% bovine serum albumin (BSA)(purified by cold ethanol precipitation), 0.1% Tween-20, and 0.1% fish gelatin in PBS (pH 7.3). Incubation of the primary antibody was carried out for 4 hours at room temperature. Following 6 washes in PBS (10 minutes each) at room temperature, the secondary antibody, an anti-rabbit antibody directly conjugated to lOnm gold particles (Vector Laboratories), was added (in blocking buffer), and allowed to react for 2 hours at room temperature. Grids were then washed 6 times (10 minutes each) in PBS at room temperature, the sample is then dried, counterstained with uranyl acetate and lead citrate, and viewed on a Phillips CM-10 electron microscope.

Identification of Tissues that Express Usherin mRNA and Protein.

Usherin is a large glycoprotein with a predicted molecular weight of 170-180 kilodaltons (Eudy et al., Science, 280:1753-1757 (1998)). The basic structure of the molecule is illustrated in FIG. 1. This figure denotes the peptides used as immunogens for the production of antibodies used in these studies, and the portions of the molecule expressed as domain-specific fusion peptides for the protein-protein interaction studies presented. The leader peptide is followed by a 300 amino acid domain with no identifiable homologies. The next 200 amino acids comprise an LN module with homology to LN domains found in the laminin family of basement membrane glycoproteins (Bruch et al., Eur. J. Biochem, 185:271-279 (1989), Yurchenco et al., Biol. Chem., 268:17286-17299 (1993)), followed by a 500 amino acid stretch containing 10 LE domains, which are rod-like laminin-EGF-like modules (Bork et al., Q. Rev. Biophys, 29:119-167 (1996); Beck et al., FASEB J., 4:148-160 (1990)), arranged in tandem. The LE domains are followed by four repeating units of about 100 amino acids each with structural homology to fibronectin type III domains. Fibronectin type III domains are shared by at least 45 different families of molecules, and are dissimilar at the amino acid level, but have very similar and identifiable tertiary structures (Sharma et al., EMBO J., 18:1468-1479 (1999)).

To test for specificity of the antibodies produced for these studies, extracts from various tissues were subjected to immunoprecipitation and western blots. In FIG. 2A, the extract was immunoprecipitated using antibody 2 or pre-immune serum from the rabbit used to raise antibody 2, and the blot developed using antibody 1. In FIG. 2B, the extract was immunoprecipitated using antibody 1 or pre-immune serum from the rabbit used to raise antibody 1, and the blot developed using antibody 2. A single band is detected of the molecular size predicted for the usherin glycoprotein (about 180 kilodaltons).

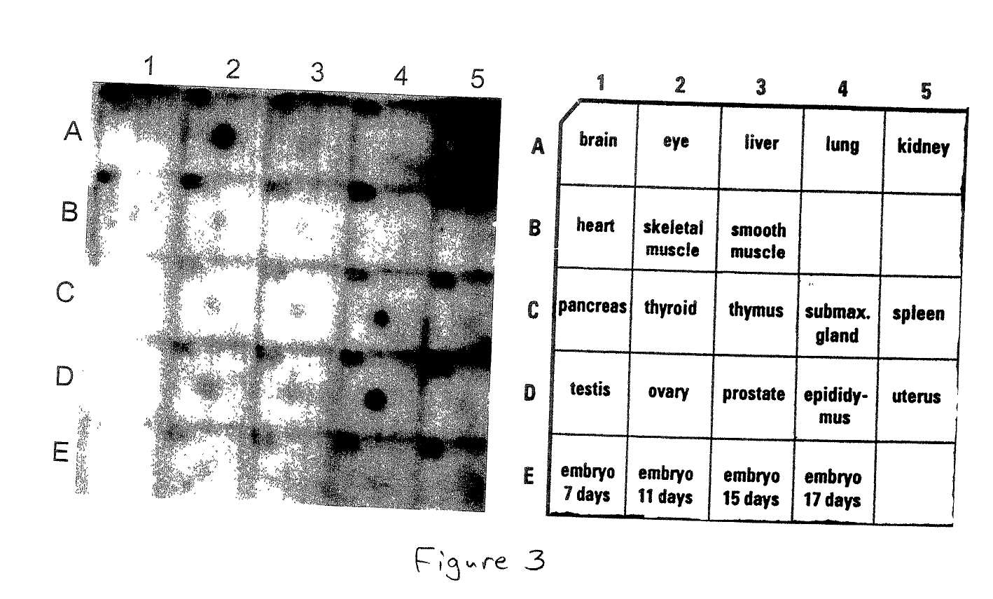

Previous results suggested usherin might have very restricted tissue distribution (Eudy et al., Science, 280:1753-1757 (1998)). Using a commercial Poly A+ RNA dot blot from mouse tissues (Clonetech), mRNA expression was identified in the ovary, epididymus, and submaxillary gland, in addition to the retina and the cochlea (FIG. 3).

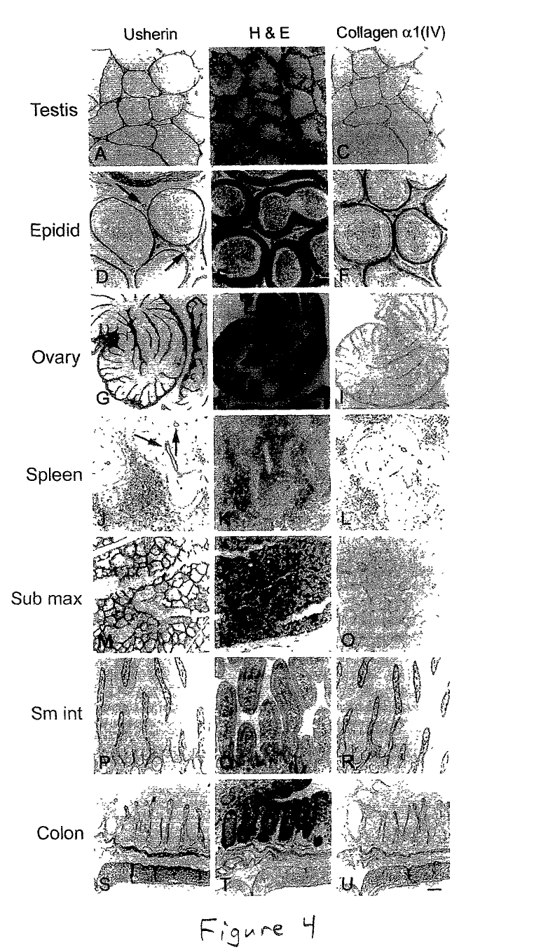

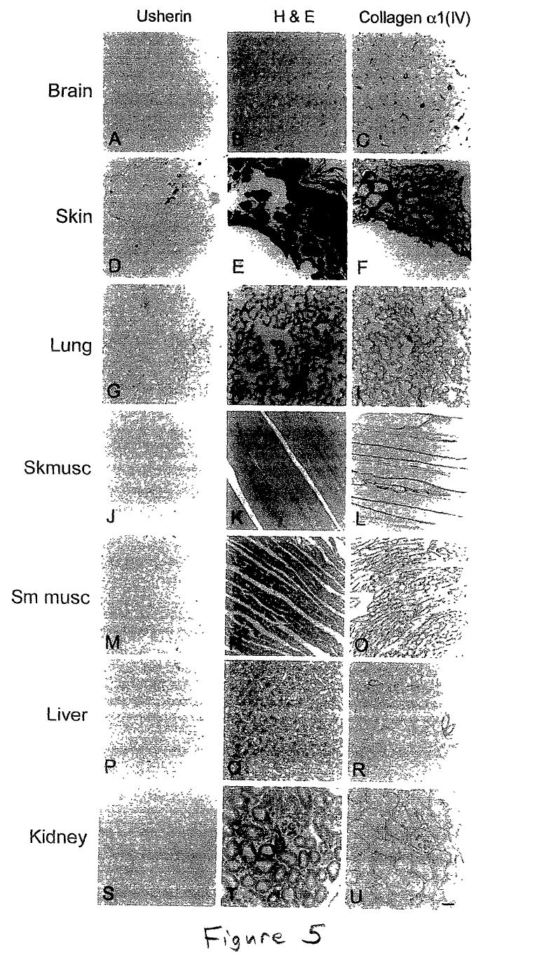

Immunohistochemical detection confirmed usherin to be expressed in the basement membranes (as inferred by co-localization with type IV collagen) of a large number of tissues, including the testis, epididymus, ovary, spleen, submaxillary gland, small intestine, and large intestine (FIG. 4). No usherin expression was detected in the brain, skin, lung, skeletal muscle, smooth muscle, liver or kidney (FIG. 5). In those tissues where usherin is expressed in structural basement membranes, it is also present in the vascular basement membranes (clearly visible in testis, epididymus and spleen (Figure, 4, denoted by arrows).

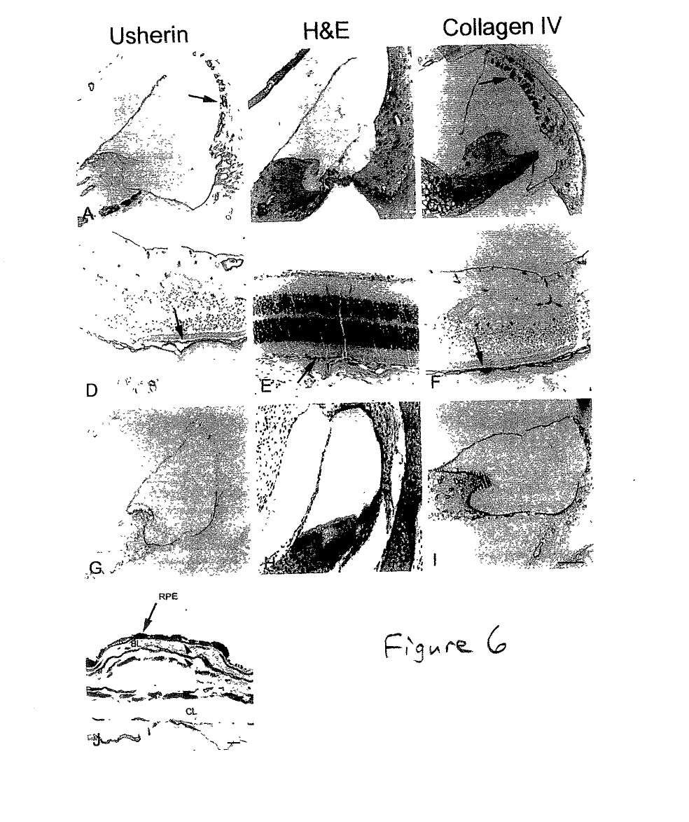

Immunohistochemical localization of usherin is illustrated for tissue sections from the retina and the cochlea, which are tissues affected in USH2A pathogenesis (FIG. 6). In the cochlea, usherin is expressed in virtually every basement membrane, as evidenced by complete co-localization with type IV collagen, which was used as a marker protein for basement membranes. Expression is particularly high in the strial capillary basement membranes (SCBM) (see arrows, FIG. 6A and C). In the retina, usherin is again expressed in all of the basement membranes, based on complete co-localization with type IV collagen (FIGS. 6D and F). It is also very prevalent in the lens capsule and the Bruch's layer between the retinal pigment epithelium and the choroid layer which is very rich in basement membranes (The Bruch's layer of the retina is denoted by arrows in FIGS. 6D and F). At postnatal day 0 (pO) in the mouse, usherin is widely expressed in the basement membranes of the cochlea (FIG. 6G). By pO in the mouse, the cells in the cochlea have not yet undergone terminal differentiation (Ehret, G., J. Am. Audio. Soc., 1(5):179-184 (March-April 1976)). The presence of usherin in the cochlear basement membranes at this time is consistent with a developmental role, as would be expected for a gene associated with a congenital deafness phenotype.

To determine whether localization is consistent from mice to humans, human retina was immunostained for the usherin. The results in FIG. 6J illustrate an immunostaining pattern consistent with the mouse. The basement membranes in the Bruch's layer and choroid capillary basement membranes are both positive for the usherin protein. Thus, in human as well as in the mouse, the retinal pigment epithelial cells lie adjacent to a basement membrane that is rich in usherin protein.

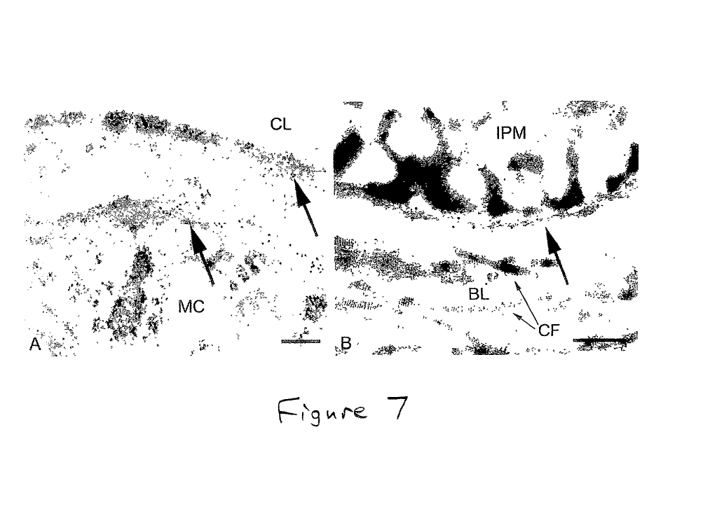

While co-localization of usherin and type IV collagen strongly suggest that usherin is a basement membrane protein, light microscopy does not provide sufficient resolution to definitively claim usherin is a basement membrane protein. Immunogold ultrastructural localization techniques were employed to establish this point. Immunogold localization using the anti-usherin antibody was performed for the cochlea and the retina. FIG. 7 illustrates that usherin clearly localizes to basement membranes in these tissues. FIG. 7A illustrates immunogold detection of usherin in the strial capillary basement membranes, and FIG. 7B illustrates immunogold localization of usherin to the basement membrane just beneath the retinal pigment epithelial cells in the Bruch's membrane of the retina. Immunogold localization confirmed basement membrane localization in all of the cochlear and retinal basement membranes examined (thus far testis, ovary, thyroid, and submaxillary gland, data not shown).

### EXAMPLE 2

### Identification of Proteins that Interact with Usherin Protein

Glutathione-S-transferase Fusion Peptides Including the Key Domains of the Usherin Protein.

From both the murine and human cDNAs, the three domains of the usherin protein (the LN domain, the LE motifs, and the fibronectin type III motifs) were amplified and sub-cloned them into the GST-fusion vector, pGEX (Pharmacia Biotech., Piscataway N.J.). The resulting fusion peptides range in molecular sizes from 45 to 46 kilodaltons (GST portion of the fusion peptide is 26 kilodaltons). Products larger than this tend to provide significantly smaller yields of recombinant protein. The precise amino acids of the usherin protein comprising the fusion products are shown in FIG. 1.

Use of Fusion Peptides to Establish Usherin Protein Interactions.

The basic procedure followed for establishing the protein interactions was as follows: Tissues were homogenized in RIPA lysis buffer (0.1% SDS, 0.5% deoxycholate, 1% Nonidet P-40, 100 MM NaCl, 10 mM Tris (pH 7.4) containing a protease inhibitor cocktail (Sigma, St. Louis, Mo.), 0.5 mM dithiothreitol (DTT), and 0.5% phenylmethylsulfonyl fluoride (PMSF). The homogenized tissues were centrifuged at 13,000 revolutions per minute (rpm) for 10 minutes at 4° C., and the supernatant was collected. To remove nonspecific binding, the supernatants were incubated with pre-immune rabbit serum and a 50% slurry of Protein A—Sepharose 4B-CL (Sigma, St Louis, Mo.) at 4° C. for 1 hour. The samples were centrifuged as above for 15 minutes, and the supernatants were retained for immunoprecipitation. Anti-sera (either GST, collagen I or collagen IV) were then added to the lysates. Samples were incubated overnight at 4° C. Then Protein A-Sepharose 4B-CL beads were added, and samples were incubated on a rocking platform for 1 hour at 4° C. The beads were pelleted by centrifugation as above for 3 minutes and washed six times with RIPA buffer containing 0.5 Molar (M) NaCl. The Protein A-Sepharose 4B CL pellet were resuspended in gel loading buffer (50 mM Tris-HCl, pH 6.8, 100 mM DTT, 0.2% SDS, 0.2% bromophenol blue, 20% glycerol), boiled for 3 minutes, and centrifuged. The immunoprecipitated proteins contained in the supernatants were electrophoretically separated using acrylamide gel and transferred onto a nitrocellulose membrane.

Protein Crosslinking.

Protein crosslinking experiments were done by using dimethyl superimidate.2HCI (DMS) (Pierce, Rockford, Ill.), following essentially the method described by Mattson et al., Mol. Biol. Rep., 17(3):167-183 (April 1993). Peptides were made to a concentration of 1 milligram per milliliter (mg/ml) in 0.1 M N-ethyl morpholineacetic acid (pH 8.5). 200 μg of each peptides were mixed in different combination. DMS was added to each peptide mix to a final concentration of 10 mM and the mixture incubated at room temperature for 60 minutes. The reaction was stopped adding ¼ volume of glacial acetic acid. The reaction mixtures were electrophoretically separated using 12% nondenaturing polyacrylamide gels. Western blots and stained gels were scanned directly and imported into Adobe Photoshop. Care was taken to duplicate the relative signal intensity of the unmanipulated data.

Identification of Proteins that Interact with Usherin.

Without being limited by a particular mechanism, it is likely that usherin, like most basement membrane proteins, is integrated into basement membranes via specific protein interactions. A fusion peptide approach was employed as a first step towards examining how usherin is integrated into the basement membrane suprastructure. This approach has the advantage of providing information regarding which domain of the usherin protein is involved in the protein interactions. The method is limited, however, in that it will not detect interactions that require post-translational modification or tertiary structural properties of the intact usherin protein. Domains (LN, LE, and fibronectin type III) of the usherin protein were expressed in Escherichia coli as a fusion product with a glutathione S-transferase (GST) tag, allowing immunoprecipitation with an anti-GST antibody, which has high specificity with minimal cross-reactivity. The basic procedure involves mixing the fusion peptides with extracts from various tissues, co-imunoprecipitating interacting proteins with anti-GST antibodies, and identifying the interacting proteins on a western blot of the immunoprecipitated material.

The LE Domain of Usherin Interacts with Type IV Collagen in Most Tissues, and the LN Domain Reacts Indirectly with Type I Collagen in Some Tissues.

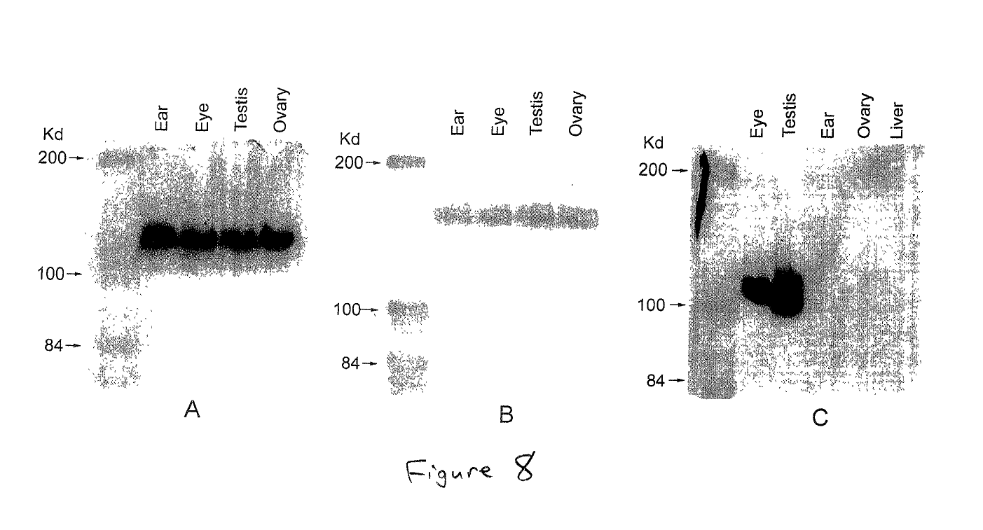

The most abundant and ubiquitous basement membrane protein is a network of type IV collagen heterotrimers comprised of α1 (IV) and α2 (IV) chains. The usherin domain-specific fusion peptides were employed in an attempt to define whether usherin interacts with type IV collagen in basement membrane. Matrix was extracted from murine cochlea, eye (following the removal of the lens) testis and ovary. The matrix extract was reacted with each of the fusion peptides comprising the domains of the usherin protein. Complexes were immunoprecipitated using the anti-GST antibody (Pharmacia Biotech., Piscataway, N.J.) and the immunoprecipitated material analyzed for type IV collagen by western blot. The data in FIG. 8A illustrates that the fusion peptide comprising the LE domain of usherin formed an immunoprecipitable complex with type IV collagen in all four tissue extracts. The type IV antibodies detect a single, band of approximately the molecular size expected for full length murine collagen α1 (IV) and α2(IV) chains (Saus et al., J. Biol. Chem., 264(11):6318-6324 (Apr. 15, 1989)). Neither the fusion peptide comprising the LN domain or the fibronectin type III domain formed a complex with type IV collagen, illustrating that the interaction between type IV collagen and usherin occurs at the LN domain of the usherin protein.

To further verify whether this interaction indeed occurs between these molecules in vivo, and is not an anomaly of the fusion peptide system, direct immunoprecipitation of the extracts was performed using antibodies against the type IV collagen a l (IV) chain. The immunoprecipitate was again subjected to western blot analysis, but probed instead with the anti-usherin antibody (antibody 2, FIG. 1). All four extracts produced a band of the correct molecular size for usherin (FIG. 8B, about 180 kilodaltons). Combined, the data in FIG. 8 provide solid evidence illustrating usherin interacts with type IV collagen via the LE domain of the usherin protein.

Based on the observation that usherin seemed to co-localize with type I collagen fibers lying adjacent to the basement membranes in the retina (set FIG. 7B) and the testis (data not shown), experiments were performed to test whether the usherin protein would specifically interact with type I collagen. Extracts were produced from retina, cochlea, testis, and ovary. Equivalent amounts of protein were reacted with the murine recombinant fusion peptides representing each of the domains and immunoprecipitated using antibodies against the GST portion of the fusion peptide. The immunoprecipitate was subjected to western blot analysis and screened using an antibody against type I collagen (Biodesign). The results in FIG. 8C illustrate that the LN domain of usherin is capable of co-immunoprecipitating type I collagen from extracts of both the testis and retina. Collagen I was not precipitated from the ear or the ovary extract, even though these tissues are rich in collagen I. This observation is consistent with an indirect interaction, where the LN domain of usherin interacts with some unknown protein(s) which interact with type I collagen. Neither the LE domain or the fibronectin type III domain immunoprecipitated type I collagen, suggesting that the protein interaction between type I collagen and usherin occurs specifically at the LN domain. This data is consistent with the immunogold localization data presented in FIG. 7B, where usherin protein is localized in a basement membrane that is in direct contact with collagen fibrils of the type I morphology. Earlier studies have confirmed that these fibrils are indeed type I collagen (Lin, W. L., Curr. Eye Res., 8(11):1171-1178 (Nov. 1989)). Similar co-localization was observed with characteristic collagen fibrils in direct contact with usherin rich basement membranes in the testis (data not shown).

Usherin Interacts with Itself, Possibly Forming an Usherin Network Within the Basement Membrane.

Interactions of usherin with other basement membrane proteins raises the question of whether usherin interacts with itself to form homodimers or possibly a homopolymeric network. To address this issue, the fusion peptide approach was again employed. In this experiment, extracts from retina, where usherin is expressed in basement membranes, and liver, which does not express usherin, were mixed with the fusion peptides, and complexed protein immunoprecipitated with anti-GST antibodies. The immunoprecipitate was subjected to western blot analysis and probed with usherin-specific antibodies. The results in FIG. 9A show that usherin forms a stable complex with the fusion peptide comprising the LN domain, but not with either the LE domain or the fibronectin type III domain.

While co-immunoprecipitation is useful for illustrating stable protein interactions, weaker interactions would not be detected. To further examine potential usherin-usherin interactions, all possible combinations of the fusion peptides were mixed and chemically cross-linked any complexes using succinyl-superimidate. The data in FIG. 9B shows that two different combinations produce cross linked products; the LE domain with the fibronectin type III (FN) domain, and the LN domain with the LE domain. In combination with FIG. 9A, these data suggest that usherin forms a stable LN-LE domain-specific complex, and a less stable LE-FN complex.

Based on the data presented in FIGS. 8 and 9, usherin is envisioned to form a network (possibly with other as of yet unidentified usherin isoforms) or sheet-like layer within the basement membrane. This layer is integrated with the type IV collage network via interaction of the LE domain of usherin with an unidentified domain of type IV collagen. Indirect tissue-specific interactions exist that link usherin to type I collagen fibrils adjacent to some basement membranes, possibly serving a structural role in those membranes.

The complete disclosures of all patents, patent applications, publications, and nucleic acid and protein database entries, including for example GenBank accession numbers and EMBL accession numbers, that are cited herein are hereby incorporated by reference as if individually incorporated. Various modifications and alterations of this invention will become apparent to those skilled in the art without departing from the scope and spirit of this invention, and it should be understood that this invention is not to be unduly limited to the illustrative embodiments set forth herein.

What is claimed is:

1. A method of determining whether an individual has or is at risk for developing Usher syndrome Type IIa, the method comprising:
obtaining a biological sample from the individual;
incubating the biological sample with at least one antibody which is immunoreactive with at least a portion of a human usherin protein under conditions effective to produce an immunoconjugate if the usherin protein is present, wherein a complement of a polynucleotide encoding the usherin protein is capable of hybridizing to the polynucleotide represented by SEQ ID NO:3 under highly stringent hybridization conditions;
evaluating for the presence or absence of the immunoconjugate; and
correlating the presence of the immunoconjugate with the individual not having Usher syndrome Type IIa, and the absence of the immunoconjugate with the individual having or being at risk for developing Usher syndrome Type IIa.

2. The method of claim 1 wherein the biological sample is selected from the groupconsisting of at least a portion of testis, cochlea, epididymus, ovary, eye, uterus, heart, pancreas, prostate, skin, placenta, spleen, submaxillary gland, small intestine, large intestine, blood vessels, and combinations thereof.

3. The method of claim 1 wherein the at least one antibody is detectably labeled.

4. The method of claim 3 wherein the detectable label is selected from the group consisting of radioactive labels, non-radioactive labels, and combinations thereof.

5. The method of claim 1 wherein the antibody is a monoclonal antibody, a polyclonal antibody, or combinations thereof.

6. The method of claim 1 wherein the antibody is immunoreactive with a polypeptide selected from the group consisting of SEQ ID NO:1, SEQ ID NO:2, SEQ ID NO:4, and combinations thereof.

7. The method of claim 1 wherein the polynucleotide encoding the usherin protein is represented by SEQ ID NO:3.

8. A method for detecting the presence or absence of an usherin protein, the method comprising:
obtaining a biological sample;
incubating the biological sample with at least one antibody which is immunoreactive with at least a portion of a human usherin protein under conditions effective to produce an immunoconjugate if the usherin protein is present, wherein a complement of a polynucleotide encoding the usherin protein is capable of hybridizing to the polynucleotide represented by SEQ ID NO:3 under highly stringent hybridization conditions;
evaluating for the presence or absence of the immunoconjugate;
correlating the presence of the immunoconjugate with the presence of usherin protein, and the absence of the immunoconjugate with the absence of the usherin protein.

9. The method of claim 8 wherein the biological sample is selected from the group consisting of at least a portion of testis, cochlea, epididymus, ovary, eye, uterus, heart, pancreas, prostate, skin, placenta, spleen, submaxillary gland, small intestine, large intestine, blood vessels, and combinations thereof.

10. The method of claim 8 wherein the antibody is detectably labeled.

11. The method of claim 10 wherein the detectable label is selected from the group consisting of radioactive labels, non-radioactive labels, and combinations thereof.

12. The method of claim 8 wherein the antibody is a monoclonal antibody, polyclonal antibody, or combinations thereof.

13. The method of claim 8 wherein the antibody is immunoreactive with a polypeptide selected from the group consisting of SEQ ID NO:1, SEQ ID NO:2, SEQ ID NO:4, and combinations thereof.

14. The method of claim 8 wherein the polynucleotide encoding the usherin protein is represented by SEQ ID NO:3.

15. A method of determining whether an individual has or is at risk for developing Usher syndrome Type IIa, the method comprising:
obtaining a biological sample from the individual;
incubating the biological sample with a first antibody and a second antibody that are immunoreactive with at least a portion of a human usherin protein under conditions effective to produce an immunoconjugate if the usherin protein is present, wherein a complement of a polynucleotide encoding the usherin protein is capable of hybridizing to the polynucleotide represented by SEQ ID NO:3 under highly stringent hybridization conditions;
evaluating for the presence or absence of the immunoconjugate; and
correlating the presence of the immunoconjugate with the individual not having Usher syndrome Type IIa, and the absence of the immunoconjugate with the individual having or being at risk for developing Usher syndrome Type IIa.

16. The method of claim 15 wherein the immunoconjugate is a sandwich comprising the first antibody, the second antibody, and the human usherin protein.

17. The method of claim 15 wherein either the first antibody or the second antibody has an attached detectable label.

18. The method of claim 17 wherein the detectable label is selected from the group consisting of radioactive labels, non-radioactive labels, and combinations thereof.

19. The method of claim 15 wherein at least one of the first or second antibody is a monoclonal antibody.

20. The method of claim 15 wherein the first antibody is a monoclonal antibody attached to a solid surface and the second antibody is a polyclonal antibody with an attached detectable label.

21. The method of claim 20 wherein the detectable label is selected from the group consisting of radioactive labels, non-radioactive labels, and combinations thereof.

22. The method of claim 15 wherein the first or second antibody is immunoreactive with a polypeptide selected from the group consisting of SEQ ID NO:1, SEQ ID NO:2, SEQ ID NO:4, and combinations thereof.

23. The method of claim 15 wherein the polynucleotide encoding the usherin protein is represented by SEQ ID NO:3.

24. A test kit for detecting the presence or absence of Usher syndrome Type IIa in an individual comprising:
an antibody that immunoreacts with at least a portion of a human usherin protein, wherein a complement of a polynucleotide encoding the usherin protein is capable of hybridizing to the polynucleotide represented by SEQ ID NO:3 under highly stringent hybridization conditions; and
a detectably-labeled usherin protein.

25. The test kit of claim 24 wherein the antibody is a monoclonal antibody, a polyclonal antibody, or combinations thereof.

26. The test kit of claim 24 wherein the antibody is attached to a solid surface.

27. The test kit of claim 24 wherein the detectable label is selected from the group consisting of radioactive labels, non-radioactive labels, and combinations thereof.

28. The test kit of claim 24 wherein the antibody is immunoreactive with a polypeptide selected from the group consisting of SEQ ID NO:1, SEQ ID NO:2, SEQ ID NO:4, and combinations thereof.

29. The method of claim 24 wherein the polynucleotide encoding the usherin protein is represented by SEQ ID NO:3.

30. A test kit for detecting the presence or absence of Usher syndrome Type IIa in an individual comprising:
a first antibody that immunoreacts with a portion of a human usherin protein; and
a second antibody that immunoreacts with a portion of a human usherin protein;
wherein a complement of a polynucleotide encoding the usherin protein is capable of hybridizing to the polynucleotide represented by SEQ ID NO:3 under highly stringent hybridization conditions.

31. The test kit of claim 30 wherein either the first antibody or the second antibody has an attached detectable label.

32. The test kit of claim 31 wherein the detectable label is selected from the group consisting of radioactive labels, non-radioactive labels, and combinations thereof.

33. The test kit of claim 31 wherein at least one of the first or second antibody is a monoclonal antibody.

34. The test kit of claim 31 wherein the first antibody is a monoclonal antibody attached to a solid surface and the second antibody is a polyclonal antibody with an attached detectable label.

35. The test kit of claim 34 wherein the detectable label is selected from the group consisting of radioactive labels, non-radioactive labels, and combinations thereof.

36. The test kit of claim 31 wherein the first or second antibody is immunoreactive with a polypeptide selected from the group consisting of SEQ ID NO:1, SEQ ID NO:2, SEQ ID NO:4, and combinations thereof.

37. The test kit of claim 31 wherein the polynucleotide encoding the usherin protein is represented by SEQ ID NO:3.

38. An antibody that immunoreacts with at least a portion of human usherin protein under conditions effective to produce an immunoconjugate if the usherin protein is present, wherein the absence of an immunoconjugate correlates to the diagnosis of or the individual being at risk for developing Usher Type IIa syndrome, and wherein a complement of a polynucleotide encoding the usherin protein is capable of hybridizing to the polynucleotide represented by SEQ ID NO:3 under highly stringent hybridization conditions.

39. The antibody of claim 38 wherein the antibody is a monoclonal antibody, a polyclonal antibody, or combinations thereof.

40. The antibody according to claim 38 wherein the antibody is immunoreactive with a polypeptide selected from the group consisting of SEQ ID NO:1, SEQ ID NO:2, SEQ ID NO:4, and combinations thereof.

41. The antibody according to claim 38 wherein the polynucleotide encoding the usherin protein is represented by SEQ ID NO:3.


SEQUENCE LISTING (4 sequences)

SEQ ID NO: 1 - protein, 23 aa, Artificial Sequence
  Immunogen
QAPPQTQGPPTVWKISPTELRIE

SEQ ID NO: 2 - protein, 201 aa, Homo sapiens
PLAQRYCIPNDAGDTADNRVSRLNPEAHPLSFVNDNDVGTSWVSNVFTNITQLNQGVTISVDLENGQYQVFYIIIQFFSPQPTEIRIQRKKENSLDWEDWQYFARNCGAFGMKNNGDLEKPDSVNCLQLSNFTPYSRGNVTFSILTPGPNYRPGYNNFYNTPSLQESVKATQIRFHFHGQYYTTETAVNLRHRYYAVDEIT

SEQ ID NO: 3 - DNA, 6330 bp, Homo sapiens
tgtttgctctgcagaatactttacctgggcaccaagtcttccttccagcattcctgctgctacagcctatttgctgagtaaccaggggttacagcagcgttgccaggcaacgagggacagcggtcctgttgaagagccatttgtcacactgaggggactggttgaaatgcaataaagaaatgataccagcagctactcatgtcttcgccattgctaagaacgtcgttggtattaccttactctgagaacgtgtctgcagtttccagaaaatggagtatcgcaacatcacttaaagtaccctgcttcaaagtattgctggcaagtggcgtgggcctgattatttatttagaaatgctttatcaggaggagaatgctttttgtaaacatgaattgcccagttctttcattgggctctggcttcttgtttcaggtcattgaaatgttgatctttgcctattttgcttcaatatccttgactgagtcacgaggtcttttcccaaggctggagaacgtgggagctttcaagaaagtttccatcgtgccaacccaagcagtatgtggactcccagaccgaagcactttttgtcacagctctgctgctgctgaaagtattcagttctgtacccagcggttttgtattcaggattgcccatacagatcttcacaccctacctacactgcccttttctcagcaggcctcagtagctgcatcacaccagacaagaatgatctgcatcctaacgcccatagcaattctgcaagttttatttttggaaatcacaagagctgcttttcttctcctccttctccaaagctgatggcatcatttaccttagctgtatggctgaaacctgagcaacaaggtgtaatgtgtgttatagaaaagacrgtagatgggcagattgtgttcaaacttacaatatctgagaaagagaccatgttttattatcgcacagtaaatggtttgcaacctccaataaaagtaatgacactggggagaattcttgtgaagaaatggattcatcttagtgtgcaggtgcatcagacaaaaatcagcttctttatcaatggcgtggagaaggatcatacacctttcaatgcaagaactctaagtggttcaattacagattttgcatctggtactgtgcaaataggacagagtttaaatggtttagagcagtttgtcggaagaatgcaagattttcgattataccaagtggcacttacaaacagagagattctggaagtcttctctggagatcttctcagattgcatgcccaatcacattgccgttgccctggcagccacccgcgggtccaccctttggcacagcggtactgcattcctaatgatgcaggagacacagctgataatagagtgtcacggttgaatcctgaagcccatcctctctcttttgtcaatgataatgatgttggtacttcatgggtttcaaatgtgtttacaaacattacacagcttaatcaaggagtgactatttcagttgatttggaaaatggacagtatcaggtgttttatattatcattcagttctttagtccacaaccaacggaaataaggattcaaaggaagaaggaaaatagtttagattgggaggactggcaatattttgccaggaattgtggtgcttttggaatgaaaaacaatggagatttggaaaaacctgattctgtcaactgtcttcagctttccaattttactccatattcccgtggcaatgtcacatttagcatcctgacacctggaccaaattatcgtcctggatacaataacttctataataccccatctcttcaagagtccgtaaaagccacgcaaataaggtttcattttcatgggcagtactatacaactgagactgctgttaacctcagacacagatattatgcagtggacgaaatcaccattagtgggagatgtcagtgccatggtcatgccgataactgcgacacaacaagccagccatatagatgcctctgctcccaggagagcttcactgaaggacttcattgtgatcgctgcttgcctctttataatgacaagcctttccgccaaggtgatcaagtttacgctttcaattgtaaaccttgtcaatgcaacagccattccaaaagctgccattacaacatctctgtagacccatttccttttgagcacttcagagggggaggaggagtttgtgatgattgtgagcataacactacaggaaggaactgtgagctgtgcaaggattactttttccgacaagttggtgcagatccttcggccatagatgtttgcaaaccctgtgactgtgatacagttggcactagaaatggtagcattctttgtgatcagattggaggacagtgtaattgtaagagacacgtgtctggcaggcagtgcaatcagtgccagaatggattctacaatctacaagagttggatcctgatggctgcagtccctgtaactgcaatacctctgggacagtggatggagatattacctgtcaccaaaattcaggccagtgcaagtgcaaagcaaacgttattgggcttaggtgtgatcattgcaattttggatttaaatttctccgaagctttaatgatgttggatgtgagccctgccagtgtaacctccatggctcagtgaacaaattctgcaatcctcactctgggcagtgtgagtgcaaaaaagaagccaaaggacttcagtgtgacacctgcagagaaaacttttatgggttagatgtcaccaattgtaaggcctgtgactgtgacacagctggatccctccctgggactgtctgtaatgctaagacagggcagtgcatctgcaagcccaatgttgaagggagacagtgcaataaatgtttggagggaaacttctacctacggcaaaataattctttcctctgtctgccttgcaactgtgataagactgggacaataaatggctctctgctgtgtaacaaatcaacaggacaatgtccttgcaaattaggggtaacaggtcttcgctgtaatcagtgtgagcctcacaggtacaatttgaccattgacaattttcaacactgccagatgtgtgagtgtgattccttggggacattacctgggaccatttgtgacccaatcagtggccagtgcctgtgtgtgcctaatcgtcaaggaagaaggtgtaatcagtgtcaaccaggtttttatatttctccaggcaatgccactggctgcctgccatgctcatgccatacaactggcgcagttaatcacatctgtaatagcctgactggtcagtgtgtttgccaagatgcttccattgctgggcaacgttgtgaccaatgcaaagaccattactttggatttgatcctcagactggaagatgtcagccttgtaattgtcatctctcaggagccttgaatgaaacctgtcacttggtcacaggccagtgtttctgtaaacaatttgtcactggctcaaagtgtgatgcttgtgttcccagtgcaagccacttggatgtcaacaatctattgggttgcagcaaaactccattccagcaacctccgcccagaggacaagttcaaagttcttctgctatcaatctctcctggagtccacctgattctccaaatgcccactggcttacttacagtttactcagggatggttttgaaatctacacaacagaggatcaatacccatacagtattcaatacttcttagacacagacctgttaccatataccaaatattcctattacattgagaccaccaatgtgcatggttcaacaaggagtgtagctgtcacttacaagacaaaaccaggggtcccagagggaaacttgactttaagttatatcattcctattggctcagactctgtgacacttacctggacaacactctcaaatcaatctggtcccatagagaaatatattttgtcctgtgcccctttggctggtggtcagccatgtgtttcctacgaaggtcatgaaacctcagctaccatctggaatctggttccatttgccaagtacgatttttctgtacaggcgtgtactagcgggggctgtttacacagcttgcccattacagtgaccacagcccaggcccctccccaaagactaagtccacctaagatgcagaaaatcagttctacagaacttcatgtagaatggtctccaccagcggaactaaatggaataattataagatatgaactatacatgagaagactgagatctactaaagaaaccacatctgaggaaagtcgagtttttcagagcagtggttggctcagtcctcattcatttgtagaatcggccaatgaaaatgcattaaaacctcctcaaacaatgacaaccatcactggcttggagccatacaccaagtatgagttcagagtcttagctgtgaatatggctggaagtgtgtcttctgcctgggtctcagaaagaacgggagaatcagcacctgtattcatgatccctccttcagtctttcccctctcttcgtactctctcaatatctcctgggagaagccagcagataatgttacaagaggaaaagttgtggggtatgacatcaatatgctttctgaacaatcacctcaacagtctattcccatggcgttttcacagctgttgcacactgctaaatcccaagaactatcttacactgtagaaggactgaaaccttataggatatatgagtttactattactctctgcaattcagttggttgtgtgaccagtgcttcgggagcaggacaaactttagcagcagcaccagcacaactgaggccacctctggttaaaggaatcaacagcacaacaatccatcttaagtggtttccacctgaagaactgaatggaccctctcctatatatcagctggaaaggagagagtcatctctaccagctctgatgaccacgatgatgaaaggaatccgtttcataggaaatgggtattgtaaatttcccagctccactcacccagtcaatacagacttcactggtaagtgtgtttgacattgctttatttaggagacacgaagctccaaaatgttttctatattttcatatccctttacaatgaatttttattatacctacttagagaaatactaattcagccctttgatagcttttgcctgattgtttcagcatgtccatctttttagaattctggggaaaaaagtcaggtaagtgaaggaaaggaaaaataaaagatgaagatgaagaagcagccttattggatcaaagtatgtgctttgtatttgtctttttgtgaagtatgtgccaggacatgtttcttgaaatattattcactgtgttctctgagcaaatgagtttgcaaaatgccctcatgctattggagattctcagtatgcaccccgttactgaaactccaaaaagcattgtaagaaagctattcaactttgcttagctaatcatgcctaacagatatttgatgtaatgttttctttttctttctcttgctgtttccttcttctttttttcactgtgacaacttaatatctcatgttctatgaagaacattgtggggaaaactaatcccagggaaaagataacttctctaagccaggactatggtaaagcaagtgaggctcttgtttcggtcacaaaatttaaaggcactaaaaaactcagtgttaatgtaaattttaatgcaatatttttaaaaatgaaaatcaatgtgaaagcactataaaaatattatcaaaagcttaaataaagacagattgaactctgtaccagcacaatcctgcctcactggccttaccctcctcctggccttactagtaccgcaatattttggaagtcccatgacctctgtgacttacagcttctaatagcatgatttcaatatagctgtaaaaaaactctacttatggtacaccatttttccaatttttaaaaaaatttacaaagtataagatatatattattatgtaaactcataaagatgttcatttaatcatccatgagaaagtcattttggagcaaatagctagtctttaaaatattgcatatgtgaagacaatgaaatggaattcgagctataaaaatttgtattgttttatttttacttaaaatagtaaatagtttgcttttcattgagactggctgctgatgcaccttggtaatgaatcatgattatattctaactgagatatattgagattaatgcatgattaactactctctcagtacatcaaaatcattgcagagtattagaaattgaaccattgagctaaaaatgctcaacttctgctttatattcttaaaatggcaaaaaaaaaaaaaaaaaa

SEQ ID NO: 4 - protein, 1551 aa, Homo sapiens
MLFVNMNCPVLSLGSGFLFQVIEMLIFAYFASISLTESRGLFPRLENVGAFKKVSIVPTQAVCGLPDRSTFCHSSAAAESIQFCTQRFCIQDCPYRSSHPTYTALFSAGLSSCITPDKNDLHPNAHSNSASFIFGNHKSCFSSPPSPKLMASFTLAVWLKPEQQGVMCVIEKTVDGQIVFKLTISEKETMFYYRTVNGLQPPIKVMTLGRILVKKWIHLSVQVHQTKISFFINGVEKDHTPFNARTLSGSITDFASGTVQIGQSLNGLEQFVGRMQDFRLYQVALTNREILEVFSGDLLRLHAQSHCRCPGSHPRVHPLAQRYCIPNDAGDTADNRVSRLNPEAHPLSFVNDNDVGTSWVSNVFTNITQLNQGVTISVDLENGQYQVFYIIIQFFSPQPTEIRIQRKKENSLDWEDWQYFARNCGAFGMKNNGDLEKPDSVNCLQLSNFTPYSRGNVTFSILTPGPNYRPGYNNFYNTPSLQESVKATQIRFHFHGQYYTTETAVNLRHRYYAVDEITISGRCQCHGHADNCDTTSQPYRCLCSQESFTEGLHCDRCLPLYNDKPFRQGDQVYAFNCKPCQCNSHSKSCHYNISVDPFPFEHFRGGGGVCDDCEHNTTGRNCELCKDYFFRQVGADPSAIDVCKPCDCDTVGTRNGSILCDQIGGQCNCKRHVSGRQCNQCQNGFYNLQELDPDGCSPCNCNTSGTVDGDITCHQNSGQCKCKANVIGLRCDHCNFGFKFLRSFNDVGCEPCQCNLHGSVNKFCNPHSGQCECKKEAKGLQCDTCRENFYGLDVTNCKACDCDTAGSLPGTVCNAKTGQCICKPNVEGRQCNKCLEGNFYLRQNNSFLCLPCNCDKTGTINGSLLCNKSTGQCPCKLGVTGLRCNQCEPHRYNLTIDNFQHCQMCECDSLGTLPGTICDPISGQCLCVPNRQGRRCNQCQPGFYISPGNATGCLPCSCHTTGAVNHICNSLTGQCVCQDASIAGQRCDQCKDHYFGFDPQTGRCQPCNCHLSGALNETCHLVTGQCFCKQFVTGSKCDACVPSASHLDVNNLLGCSKTPFQQPPPRGQVQSSSAINLSWSPPDSPNAHWLTYSLLRDGFEIYTTEDQYPYSIQYFLDTDLLPYTKYSYYIETTNVHGSTRSVAVTYKTKPGVPEGNLTLSYIIPIGSDSVTLTWTTLSNQSGPIEKYILSCAPLAGGQPCVSYEGHETSATIWNLVPFAKYDFSVQACTSGGCLHSLPITVTTAQAPPQRLSPPKMQKISSTELHVEWSPPAELNGIIIRYELYMRRLRSTKETTSEESRVFQSSGWLSPHSFVESANENALKPPQTMTTITGLEPYTKYEFRVLAVNMAGSVSSAWVSERTGESAPVFMIPPSVFPLSSYSLNISWEKPADNVTRGKVVGYDINMLSEQSPQQSIPMAFSQLLHTAKSQELSYTVEGLKPYRIYEFTITLCNSVGCVTSASGAGQTLAAAPAQLRPPLVKGINSTTIHLKWFPPEELNGPSPIYQLERRESSLPALMTTMMKGIRFIGNGYCKFPSSTHPVNTDFTGKCV